(19) United States
(12) Patent Application Publication
(10) Pub. No.: US 2012/0231517 A1
(43) Pub. Date: Sep. 13, 2012

(54) MINIATURIZED ELECTROPORATION-READY MICROWELL ARAY FOR HIGH-THROUGHPUT GENOMIC SCREENING

(75) Inventors: Enrique Saez, San Diego, CA (US); Tilak Jain, Encinitas, CA (US)

(73) Assignee: The Scripps Research Institute, La Jolla, CA (US)

(21) Appl. No.: 13/261,260

(22) PCT Filed: Oct. 13, 2010
(86) PCT No.: PCT/US2010/052501

§ 371 Date: May 30, 2012

Related U.S. Application Data
(60) Provisional application No. 61/251,086, filed on Oct. 13, 2009.

(51) Int. Cl. C12N 13/00; G03F 7/20; C12M 1/42; B82Y 5/00

(52) U.S. Cl. 435/173.6; 435/283.1; 430/319; 977/773; 977/904

(57) ABSTRACT

Methods of introducing exogenous molecules into cells including cell lines and primary cells are provided. Additionally, miniaturized electroporation-ready microwell arrays are provided. These tools provide a miniaturized high-throughput functional genomics screening platform to carry out genome-size screens in a variety of cell types.

### CROSS-REFERENCE TO RELATED APPLICATION

This application claims the benefit of U.S. Provisional Patent Application Ser. No. 61/251,086, filed Oct. 13, 2009, entitled “MINIATURIZED ELECTROPORATION-READY MICROWELL ARRAY FOR HIGH-THROUGHPUT GENOMIC SCREENING,” the entirety of which is incorporated herein by reference.

### BACKGROUND

High-throughput cell-based screens of genome-size collections of cDNAs and siRNAs have become a powerful tool to annotate the mammalian genome, enabling the discovery of novel genes associated with normal cellular processes and pathogenic states, and the unraveling of genetic networks and signaling pathways in a systems biology approach. However, the capital expenses and the cost of reagents necessary to perform such large screens have limited application of this technology.

Efforts to miniaturize the screening process have centered on the development of cellular microarrays created on microscope slides that use chemical means to introduce exogenous genetic material into mammalian cells. While this work has demonstrated the feasibility of screening in very small formats, the use of chemical transfection reagents (effective only in a subset of cell lines and not on primary cells) and the lack of defined borders between cells grown in adjacent microspots containing different genetic material (to prevent cell migration and to aid spot location recognition during imaging and phenotype deconvolution) have hampered this screening technology.

Thus, functional annotation of the mammalian genome has proven particularly difficult. Unlike bacteria, yeast, C. Elegans, Drosophila and other lower organisms, where large genetic screens can be carried out with relative ease to discern gene purpose, tools to delineate gene function, signaling pathways and genetic networks in mammalian cells have been very limited. The recent discovery and application of RNA interference (RNAi) gene silencing technology has dramatically expanded the ability of biologists to perturb gene function in mammalian cells on a global scale¹⁻⁵. Together with screens of large cDNA collections, genome-wide RNAi screens recently completed in mammalian cells are yielding a wealth of new gene annotations^{6,7}. These high-throughput cell-based screens use 96 or 384-well plate platforms that require expensive reagents and extensive usage of robotics for plate manipulation, liquid handling, and assay. To fully realize the potential of cell-based genetic screening in mammalian cells it is imperative that an advanced screening technology platform be developed.

A substantially miniaturized ‘reverse transfection’ method for parallel chemical transfection of cDNAs into mammalian cells cultured on a glass microscope slide has been described⁸. Plasmids encoding various cDNAs were arrayed onto glass slides using a standard microarraying robot and then incubated with a lipid transfection reagent. The slides were placed in a tissue culture dish, and cells were seeded on top. As the cells sat on the DNA spots, they took up the underlying DNA (reverse transfection). Phenotypic analysis was typically carried out by fixing the cells and visualizing the results using a microarray scanner or an automated scope. Using this cell microarray format, ˜5,000 genetic experiments could be conducted on a single microscope slide⁹. Modification of the surface chemistry of the slide also allowed the development of cell microarrays of non-adherent cell lines¹³.

These approaches share three critical weaknesses that drastically limit application of this technology. First, they rely on chemical transfection to deliver the nucleic acid of interest into cells. This precludes the use of hard-to-transfect cell lines and virtually all clinically relevant primary cells (not transfectable with chemical reagents). Second, cell microarrays lack physical barriers to contain cells transfected with one nucleic acid from migrating and mixing with cells transfected with another nucleic acid. Migration of cells between spots can lead to inter-spot contamination, confounding phenotypic analysis and hindering time-lapse studies (where spots are visited multiple times over the course of the assay). Third, because the cells grow in a lawn without reference points, it is difficult to identify with certainty the microscale regions corresponding to individual spots during automated image analysis, and minor errors in imaging (due to tolerances in the microscope stage or during microarraying) can lead to incorrect phenotypic annotations.

### SUMMARY OF THE INVENTION

The invention relates to the introduction of exogenous molecules into cells. In some embodiments, the described materials and methods provide tools for high-throughput cell-based screens of exogenous genetic material. The invention is also directed to microwell arrays on an electroporation-ready substrate and procedures to achieve highly efficient parallel introduction of exogenous molecules into human cell lines and primary mouse macrophages. The microwells confine cells and offer multiple advantages during imaging and phenotype analysis. The invention is further directed to a method to load the described microwell arrays with libraries of nucleic acids using a standard microarrayer. These tools of the invention form the basis of a miniaturized high-throughput functional genomics screening platform to carry out genome-size screens in a variety of mammalian cells.

### BRIEF DESCRIPTION OF THE FIGURES

FIG. 1: Electroporation (EP) of exogenous molecules into HEK 293T cells growing on ITO without microwells. A) Phase contrast, transfection assay (Propidium Iodide fluorescence 2 hrs post-EP) and viability assay (Calcein AM 1 day post-EP) images for three different electroporation parameter sets (electric field intensities 100 V cm⁻¹, 500 V cm⁻¹ and 800 V cm⁻¹ respectively, at constant pulse width of 1 ms and 1 square pulse). A scratch (not shown) was made on each of the ITO pieces to locate the culture area for the two assays at different time points. B) Further confirmation of viability post-EP. Cells electroporated with propidium iodide (top row: red) using the optimal electroporation parameter (500 V cm⁻¹, pulse width 1 ms and 1 square pulse) were virally transduced with viral-GFP particles and assayed for viability 24 hr post-EP (bottom row: green). GFP expression is a confirmation of cell viability. C) Electroporation of Alexa Fluor 488-conjugated siRNA molecules using two electroporation parameter sets (100 V cm⁻¹ and 500 V cm⁻¹; constant pulse width of 1 ms and 1 square pulse) and assaying for transfection using Alexa Fluor 488 compatible excitation/emission filters 2 hr post-EP.

FIG. 2: Schematic showing the step-by-step process from creation of microwell arrays to image analysis after electroporation. A) Bonding of a pre-cleaned ITO slide with a laser cut microwell array stencil/coverlay. B) Sterilization and coating with fibronectin to enhance cellular adhesion. C) Seeding of mammalian cells into the microwell array by placement of the substrate and cells in a tissue culture dish. 1 hr after seeding, the dish was gently washed to remove unbound cells. Cells inside the microwells experience minimal flow stress and remain attached during this step. D) Electroporate substrate using either a single or double cathode scheme. Post-electroporation incubation. E) Image acquisition of either single microwells under a microscope or whole slide scanning.

FIG. 3: Culture and electroporation of HEK 293T cells in microwell arrays on ITO coated glass slides. A) Culture of HEK 293T cells within individual microwells 1 day after seeding. Top row: Phase contrast image of cells. Bottom row: live assay of cells using Calcein AM within microwells. B) Electroporation of HEK 293T cells growing within microwell arrays. Electroporation parameter set used: 500 V cm⁻¹, 1 ms pulse-width and 1 square pulse. Top row: brightfield image of cells post-electroporation. Bottom row: fluorescence image of cells at appropriate molecule compatible excitation/emission spectra. Left column: electroporation of propidium iodide. Middle column: electroporation of Alexa Fluor 488-labeled siRNA. Right column: electroporation of plasmid encoding GFP. C) Estimation of transfected cell count in an individual microwell using software identification of the microwell edges as physical markers for identification of microscale culture spatial locations.

FIG. 4: Electroporation of primary macrophage cells within microwells and automated image analysis of microcultures. A) Electroporation of primary macrophages within microwell arrays. All images taken at 2 hr post-electroporation. Left column: Phase contrast image post-EP. Middle column: Transfection assay using Propidium Iodide post-electroporation. Right column: Live assay with Calcein AM post-EP. Top row: Control pulse (CP) 100 V cm⁻¹, 1 ms pulse-width and 1 square pulse. Bottom row: Electroporation pulse (EP) 600 V cm⁻¹, 1 ms pulse-width and 5 square pulses at 1 Hz. B) Automated microwell edge detection and image analysis estimating total cell nuclei (Hoechst), transfected cells (Propidium Iodide) and viable cells (Calcein AM) in individual microwells of the images shown in FIG. 4A.

FIG. 5: Effect of electrode configuration on electroporation efficiency variation across microwell array substrate. A) Finite Element Analysis FEMLAB (Comsol, Calif.) simulations of electric field across a 484 microwell array on a conductive microscope slide using either a single or double cathode configuration. B) Analysis of mean and standard deviation, as a result of the simulations, by binning electric fields at the center of each microwell of the array using the single or double cathode configuration. Individual columns are binned at 10 V cm⁻¹. Eₜ shows the estimated threshold of electric field required for ˜50% electroporation efficiency relative to maximum as determined by matching experimental data to simulated electric field values. C) Parallel electroporation of mammalian cells in 400 microwells of the array (the outermost microwells of the 484 microwell array were excluded to avoid potential edge effects) with propidium iodide using previously optimized parameters with the single cathode configuration or D) double cathode configuration. Insets on the left and right show a zoomed out 4×4 array from the left and right sides of the larger 484 microwell array. Bar graphs indicate electroporation efficiency (measured as relative fluorescent units, RFU, of internalized propidium iodide) using a single or double cathode configuration. Individual bars represent mean values across the 20 rows of a single column plotted left to right of the microwell array. E) Cell viability and transfection efficiency of a 400 microwell array 1 hr post-electroporation, with a double cathode set up. The adjacent plots on the top and side of each image indicate fluorescence intensity in the central row and column in each case.

FIG. 6: Parallel electroporation of Alexa 488 fluor siRNA into HEK 293T cells contained within the microwell array. Electroporation was conducted using the double cathode method with an electric field intensity of 500 Vcm⁻¹, 1 ms pulse-width and 1 square pulse. Two hours post-electroporation, the cells were washed in PBS, fixed and scanned for green fluorescence on a ProScanArray HT confocal laser slide scanner (Perkin Elmer, MA). Prior to scanning, the microwell stencil was removed with forceps to eliminate auto-fluorescence from the stencil material. The artifact in the lower-left corner is due to handling with forceps.

FIG. 7: Microarraying within microwells using an iterative process of imaging and calibration. A) A blank slide is spotted with printing buffer to determine the X-Y offset error from a microwell array slide to be printed on. Both slides are independently scanned and their images overlaid in software to determine the offset. B) After re-calibrating the microarrayer with the offset error, Alexa Fluor 488-labeled siRNA was spotted directly into the microwell array.

FIG. 8: A) Flow chart depicting the protocol for optimization of electroporation parameters on ITO glass pieces. B) Custom-built electroporation setup used for the optimization protocol. C) “Browning curve.” Electroporation parameter sets which cause browning on the ITO substrate. The browned ITO usually caused cells to detach from the surface and therefore was avoided during optimization. The area below the curve was used during the optimization of electroporation parameters (voltage and pulse-width). D) Image of a “browned” ITO coated glass piece.

FIG. 9: HeLa cells were seeded on ITO pieces and optimization of electroporation parameters was carried out by varying the voltage and pulse-width. Representative images are shown for three different electroporation parameter sets. All images taken post-electroporation (post-EP). Left column: Phase contrast image (2 hr post-EP). Center column: Transfection assay with Propidium Iodide (2 hr post-EP). Right column: Calcein live assay (1 day post-EP).

FIG. 10: illustrates a microfabricated 2,592 microwell array. (a) Design schematic. (b) Finished prototype. (c,d) sections of array. (e) Electroporation of Alexa-488 fluor labeled siRNA (bottom, in green) into cells grown on prototype with high efficiency.

FIGS. 11A-C: Schematic of high-throughput screening in the miniaturized technology platform. FIG. 11A) Top view of microwell arrays. Libraries of molecules are mapped/loaded from stock plates into segregated microwells in the miniaturized platform. Cells are seeded into the microwells and the loaded molecules are electroporated. The microwell arrays with cells are incubated in media, assayed, imaged and analyzed to screen for hits. FIG. 11B) Cross-sectional view of microwell arrays. Microwell arrays are fabricated and prepared for loading libraries of molecules. Liquid handling equipment (such as pin-based microarrayers or piezo-electric dispensers) is used to load the molecules in addressable microwells. Cells are then seeded within the microwells and electroporation is carried out. Cells within specific microwells are electroporated with only the existing molecules loaded in that microwell. FIG. 11C) Schematic showing two different methods to bind and release exogenous molecules in the microwells during cell seeding and prior to their electroporation. Left column: surface charges (positive in this case) can be used to bind the exogenous molecules electrostatically. Upon cell seeding the molecules slowly diffuse from the surface and are henceforth electroporated into the overlaying cells. Right column: exogenous molecules are loaded with biodegradable/dissoluble release agents. Upon cell seeding the release agent degrades/dissolves, resulting in high concentration of free molecules inside the microwell, which are then electroporated.

FIG. 12A: Examples of microfabrication processes used to fabricate a microwell array are illustrated.

FIG. 12B: Illustrates examples of the electric fields created in a microwell array during electroporation.

FIG. 13: Electroporation in microfabricated microwells with molecules suspended in electroporation buffer. A) Top row shows microwells containing Human Embryonic Kidney (HEK) 293 cells electroporated with Propidium Iodide (small molecule) and nuclei stained. As shown, cells strongly fluoresced blue. A viability assessment post-electroporation was done with Calcein AM dye, showing cell viability was high. Bottom row shows part of a microwell array electroporated with Alexa-488 fluor conjugated siRNA molecules (nucleic acid). B) Electroporation of siControl (scrambled siRNA designed to target no known human RNA) and siRPS27a (siRNA designed to target and knock down Ribosomal Protein Subunit 27a (RPS27a), leading to cell death due to insufficient translation). Forty-eight hours later siControl electroporated microwell arrays show high cell viability, whereas siRPS27a electroporated microwell arrays show increased cell death. C) Plasmid DNA encoding green fluorescent protein (GFP) electroporated into HEK 293 cells and imaged 48 hours post-electroporation, in comparison to microwells where no electroporation pulse was applied. As shown, GFP expression was high in electroporated cells compared to non-electroporated cells. Scale: Microwells are 400 micron square dimensions and separated by 500 microns.

FIG. 14: Loading of microwells with exogenous molecule. Two different equipment types can be used to accomplish precisely aligned molecule loading in microwells. A) Schematic of molecule loading using a contact pin-based microarrayer equipment. The tip delivers the solution containing the molecule by touching the bottom surface of the microwell. B) Schematic of molecule loading using a non-contact piezo-electric dispenser equipment. The head dispenses sufficient solution containing the exogenous molecules to load the microwell. C, D) Example of aligned loading fluorescent-conjugated siRNA molecules in microwell arrays using either the contact pin microarrayer or piezo-electric dispenser (dispense volume per microwell was 10 nL) respectively. Microwells are 400 micron square dimensions and 500 micron separated.

FIG. 15: Electroporation of functional molecules (siRNA) loaded in microwells in a pre-determined layout. As shown in the schematic on the left, diagonal quadrants were loaded with siControl (scrambled siRNA designed to target no known human RNA) and siRPS27a (siRNA designed to target and knock down Ribosomal Protein Subunit 27a (RPS27a), leading to cell death due to insufficient translation). Human Cervical Carcinoma HeLa cells were seeded on the microwell array and electroporated with a single 70 V and 1 ms pulse (anode placed 1 mm from ITO surface cathode). Phenotypes assessed 48 hrs post-electroporation show cell death specifically in microwells located in quadrants where siRPS27a molecules were loaded.

FIG. 16: Enhancement of cell containment within microwells using poly-ethylene glycol molecules (PEG) bound on microwell walls. Top: schematic of PEG molecules bound only on the microwell wall material, but not on the bottom surface of the microwell. Bottom: Human Embryonic Kidney cells (HEK) 293 cells were seeded on microwell arrays and demonstrated higher containment in microwells 1 day post-seeding with the presence of PEG.

### DETAILED DESCRIPTION OF THE INVENTION

### Definitions

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.
As used herein, the term “about” is used to refer to an amount that is approximately, nearly, almost, or in the vicinity of being equal to a stated amount. The terms “about” and “approximately” are used interchangeably throughout this document.
As used herein, the phrase “consisting essentially of” limits a composition to the specified materials or steps and those additional, undefined components that do not materially affect the basic and novel characteristic(s) of the composition.

References in the specification to “one embodiment,” “an embodiment,” “an example,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, the terms “transfect” and “transfection” mean the introduction of an exogenous molecule into a cell. Any method that causes an exogenous molecule to be introduced into a cell is a transfection method. Any exogenous molecule can be transfected into a cell, including, but not limited to, amino acids, bioactive molecules, polypeptides, nucleic acids, RNA, DNA, viruses, drugs, and nanoparticles as described in detail elsewhere in this document. As used herein, “transfect” and “transfection” are meant to include, but are not meant to be limited to, transduction and transformation.

As used herein, the term “electroporation,” also known as electropermeabilization, is the introduction of an exogenous molecule into a cell by use of a significant increase in the electrical conductivity and permeability of the cell plasma membrane caused by an externally applied electrical field.

A genome-wide mammalian genomic screening platform is provided according to the invention. This platform provides dramatically reduced screening costs, ease of use, the ability to run screens multiple times to enhance data quality, ease of storage of microfabricated screening substrates, and the ability to avoid chemical transfection. The platform allows the use of prokaryotic cells, eukaryotic cells, single-celled organisms, human cells, primary cells and difficult to transfect cell lines. According to the invention, certain embodiments use electroporation to introduce nucleic acids into mammalian cells. Also, certain aspects of the invention provide the fabrication of a microwell array to create spatially defined regions of microscale cultures and to restrict cell motility. In preferred embodiments according to the invention, the edges of the microwells allow for accurate determination of microscale culture position on the conductive substrate during image acquisition and phenotypic analysis.

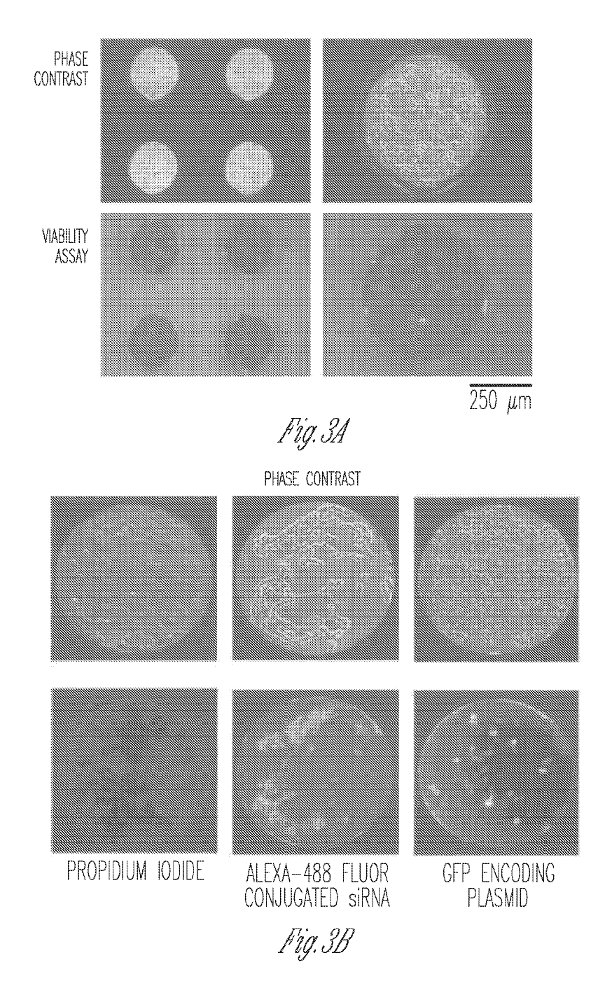

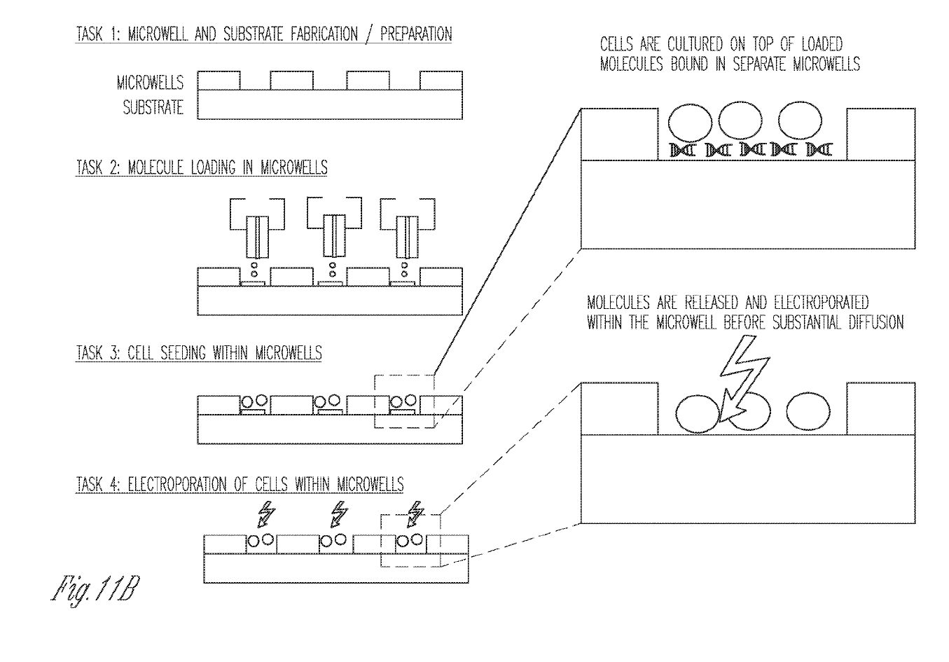

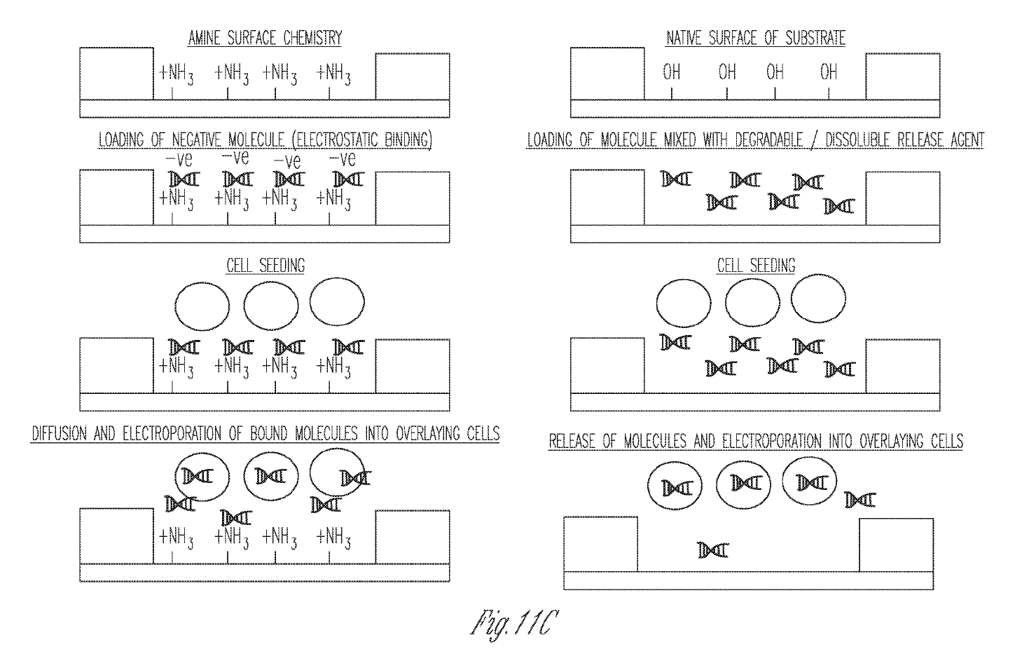

FIGS. 2 and 11B illustrate examples of a method of parallel introduction of nucleic acids into mammalian cells. This method uses electroporation to introduce the nucleic acids into mammalian cells. Additionally, this method uses a microwell array to create spatially defined regions of microscale cultures and to restrict cell motility. The edges of the microwells additionally allow for accurate determination of microscale culture position on the conductive substrate during image acquisition and phenotypic analysis, an essential component of a high-throughput genetic screening platform. Sections A through E below further elaborate on these aspects of the invention as shown in FIGS. 2 and 11B.

### Section A—Microwell Array Creation

Section A of FIG. 2 illustrates one example of a microwell array for containing cells. In this example, the microwell array is created by mounting a walled frame onto a substrate. The substrate can be any conductive material, including transparent, translucent, and opaque materials. The conductive material portion bonded to the base portion may be contiguous, such as a layer, or non-contiguous, such as a pattern. A transparent substrate is preferred. In some embodiments, transparent substrates are employed in conjunction with inverted microscopy for assay analysis. In other embodiments, an opaque substrate (such as stainless steel, gold, platinum, or doped silicon) is employed. In some embodiments, an opaque substrate is employed in conjunction with bright fluorescence or non-fluorescent methods such as luminescence or colorimetric methods, for image acquisition. As shown in FIG. 2, the substrate comprises an optically transparent conductive material bonded to a base material. In the examples described herein, the base material is glass. Additionally, as described herein, indium-tin oxide (ITO) may be used as the transparent conductive material. The ITO is bonded to one side of the glass base. In other embodiments, however, other conductive materials, such as graphene, are used.

The microwells can be created on all types of substrates. In some embodiments, the substrate is composed of different layers (components). In certain embodiments, the substrate is rigid. In other embodiments, the substrate is flexible. In certain embodiments, glass is employed as a base support and a conductive metal is deposited thereon; thus, the two layers provide separate functions. Glass provides the base structure, whereas the conductive metal provides the conductivity. Borosilicate, quartz, soda-lime, and both porous and non-porous glass are contemplated for substrate base use. In alternative embodiments, a metal such as stainless steel is used as the base and for conductivity. In some embodiments, the base support is composed of flexible plastic, silicon, polysilicon, doped silicon or Teflon and the conductive material is a conductive polymer or a gold film. In some embodiments, a composite material containing conductive particles is employed for the base portion. In some embodiments, the substrate base is made of plastic or polymer. Poly-methyl-methacrylate (PMMA), Polystyrene, Polycarbonate, and Prolypropylene are all contemplated for the substrate base material. In some embodiments, the substrate base is made of metal or ceramic. Microwells can be of any shape, for example squares, circles, rectangles or any other desired shape.

As mentioned above, the walled frame is mounted onto the substrate to create the microwell array. The walled frame comprises an optically visible material having an array of apertures separated by walls. The walled frame is optically visible such that the walls of the walled frame can be identified by an optical camera. In one example, the walled frame is formed of photo-definable biocompatible polymers. The microwells are formed when the walled frame is placed onto the substrate. Each aperture of the walled frame forms the walls of a microwell and the surface of the substrate forms the bottom of the microwell. Accordingly, each microwell is separated from adjacent microwells by a wall of the walled frame. In some examples the microwell array contains 484 microwells. In some examples the microwell array contains 2,592 microwells. In some examples, the microwell array is expanded to contain 20,736 microwells, which is approximately human genome capacity. In some examples, the microarray is housed in a standard 96 or 384 well plate footprint. The microwell array is mounted onto the side of the substrate having the conductive material (ITO) thereon. Accordingly, the conductive material is located on the bottom of each microwell.

In one example, the microwell array is created by placing a laser cut coverlay on the center of a substrate. One example of a suitable coverlay is the FlexTac BGA Rework Stencil 22×22 array having a thickness of 100 μm (produced by CircuitMedic, MA). In one example, the walled frame is mounted to the substrate with an adhesive. For example, the coverlay is adhered to a conductive ITO coated microscope slide using the pre-coated adhesive provided on the backside of the coverlay. In other examples, to obtain precise straight edges and defined geometric features, microfabrication techniques using photopatternable polymers are used to create the walled frame.

In the example shown in FIG. 2, the walled frame comprises an array of 484 individual apertures wherein each aperture is a 500 μm diameter cylindrical aperture and is separated from adjacent apertures by at least 1 mm. In this example, the dimensions of the microwells are selected to ensure that enough cells can be accommodated per microwell to assess phenotypes with statistical power. In other examples, other sizes of microwells and/or distances between microwells are used. In some examples, the height of the microwells can be increased to minimize cell migration and flow stress on the cultures seeded in the microwells. In examples described herein, the height of the microwells for the 484 well array is 100 microns, while the height of the microwells for the 2592 well array is approximately 25 microns.

In one example, a pattern of a highly conductive material (e.g. gold) is overlaid on the conductive material (ITO) on the substrate. This pattern of highly conductive material is also referred to herein as an electrode grid. The electrode grid is used to increase the uniformity of the electric field in some examples. Another function of the microwell is as an insulator for the electrical pattern. In some embodiments, the electrode grid (e.g., the electrical conducting pattern), whose primary function is to distribute the potential across the conductive layer, should not come in direct contact with the electroporation buffer. Insulation prevents any flow of current directly from the electrode grid (e.g., the pattern) to the buffer. The insulation can be accomplished by patterning another insulation layer that overlays the electrode grid (e.g., the patterns) or by using the microwell array itself as the insulator. In one example of a 2592 well array, the photo-polymer is used as both the microwell layer and the insulating layer for the underlying electrode grid (e.g., the electrical gold pattern).

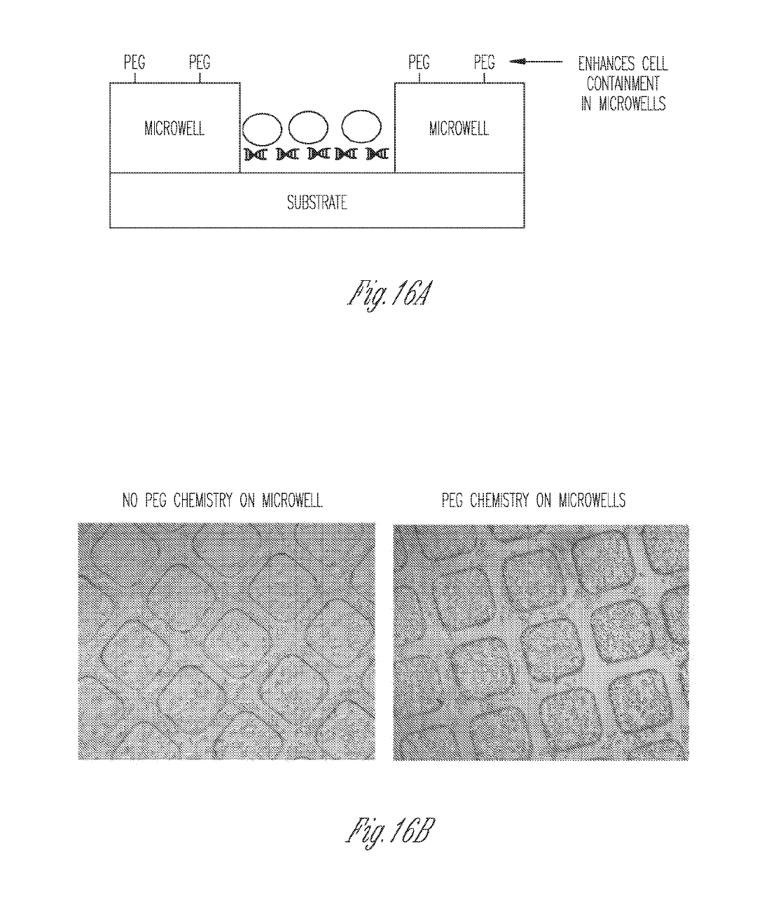

Microwells offer numerous advantages for a miniaturized genomic screening platform. They can provide a physical marker for imaging and a barrier for microscale cultures to be contained. The use of microwells significantly enhances the ability to identify the spatial location of microscale cellular cultures and improves the image acquisition and analysis steps. The microwell edges also provide a clear physical indication of the spatial location of the cultures and they enable centering of individual microscale images during image processing and analysis. Moreover, the microwells provide physical containment for cells transfected with an individual nucleic acid, restricting migration and contamination of neighboring cellular cultures transfected with other nucleic acids. This feature may be particularly relevant for time-lapse studies, in which cells are monitored multiple times after transfection. In some embodiments, the surface chemistry of the microwell walls is modified to repel cells. This aids in separating cells in one microwell from cells in another microwell (see FIG. 16). Another advantage of microwells is that microscale cultures experience significantly lower flow shear stresses as indicated by simulations. This may minimize cell stripping during experimental protocols that could lead to inter-spot contamination.

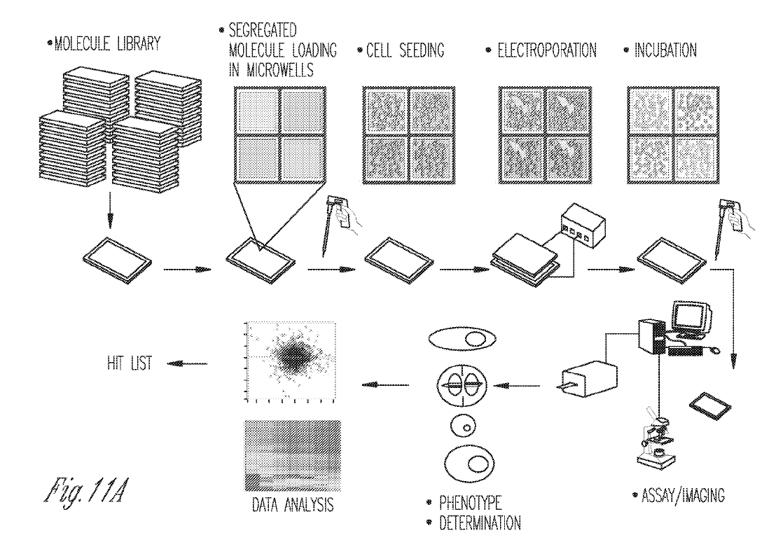

FIG. 10 illustrates another example of a microwell array for containing cells. The microwells are fabricated on a transparent conductive Indium-Tin oxide (ITO) coated glass substrate and are 400 μm×400 μm×50 μm (L×W×H) with an inter-well distance of 500 μm. These dimensions ensure that each microwell can accommodate 200-800 cells (depending on cell type), providing the statistical power to conclusively evaluate phenotypes during a screen. A 50 μm height is sufficient to restrict cell motility between microwells. Using these microwell dimensions and spacing, 20,736 microwells can be fabricated on a substrate of dimensions 75 mm×75 mm (144 columns×144 rows) that can then be housed on a standard 96/384 well footprint (85.48 mm×127.76 mm) well-suited for use with available image analysis systems. This microwell spacing is compatible with existing microarrayers and will allow loading of genome-scale siRNA libraries.

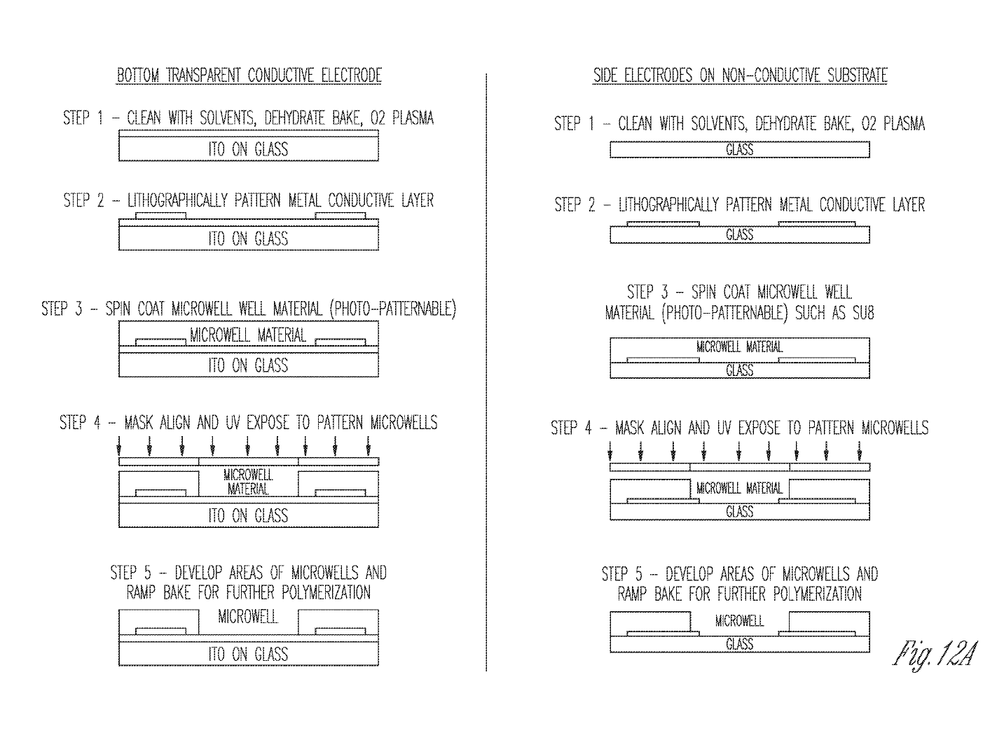

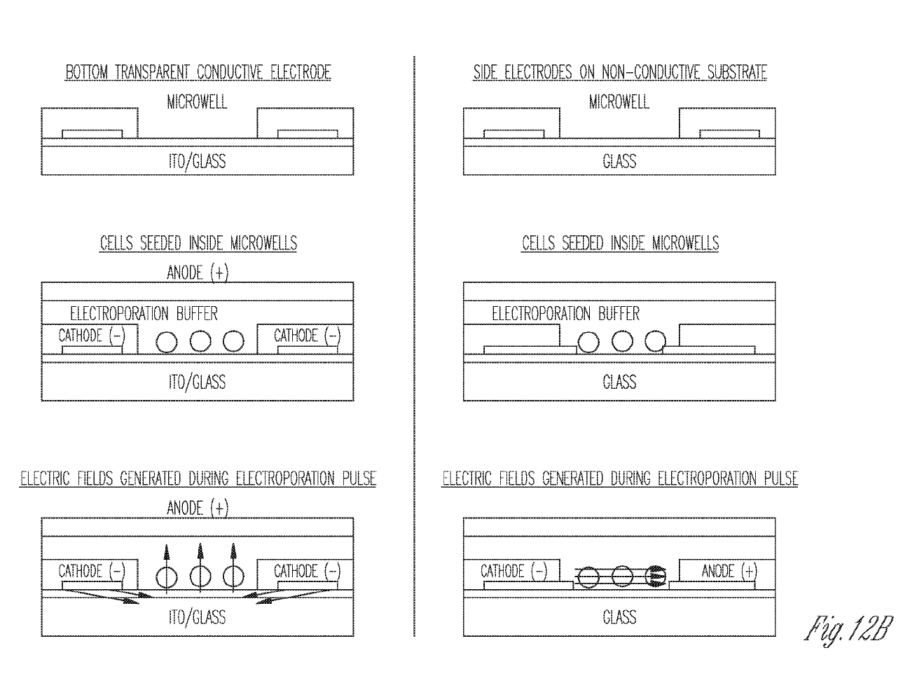

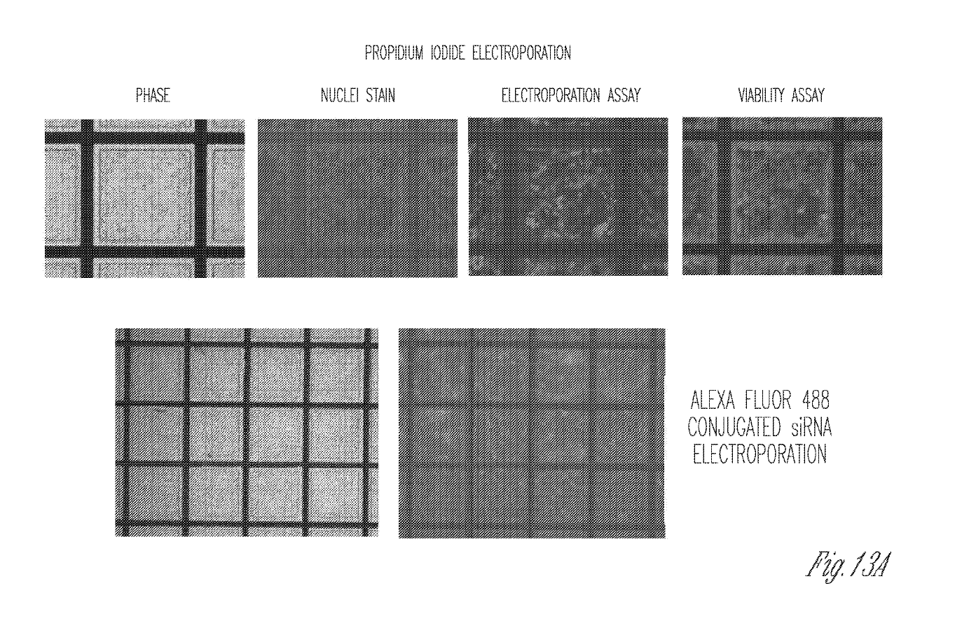

FIG. 12A illustrates example methods for fabricating a microwell array. In particular, FIG. 12A illustrates example microfabrication processes used to fabricate an electrode layer on a substrate of a microwell array. FIG. 12A illustrates two different examples: the first (illustrated in the left column) creates a microwell array having an electrode layer on a conductive portion of a substrate, and the second (illustrated in the right column) creates a microwell array having an electrode layer on a non-conductive portion of a substrate.

### Example 1

### Bottom Electrode Microwell Array

Example 1 begins with a substrate including a conductive material bonded on top of a base material. In an example, the base material is non-opaque (e.g., transparent or translucent). The base material can be either flexible or rigid and either conductive or non-conductive. For example, the base material can include a glass including borosilicate, quartz, soda-lime, and porous or non-porous glass. In other examples, the based material can include a plastic (e.g., a polymer) including Poly-methyl-methacrylate (PMMA), Polystyrene, Polycarbonate, and Polypropylene. In another example, the base material is opaque and can be a silicon, metal, ceramic and the like. In examples where the base material is conductive, the base material can include, for example, a metal, polysilicon, doped silicon, metal alloys, and composite materials containing conductive particles.

The conductive material can be bonded to the base material and can provide a bottom electrode (e.g., the cathode) used in electroporation. The conductive material can cover substantially all of a top surface of the base material or can be a pattern where portions of the top surface of the base material are not covered by the conductive material. In an example, the conductive material is non-opaque (e.g., transparent or translucent). For example, the conductive material can include Indium Tin Oxide (ITO) or graphene. In another example, the conductive material is opaque.

In an example, an electrode layer is formed on the substrate. In some examples, the electrode layer provides greater electrical conductivity than the conductive material of the substrate. Thus, the electrode layer can improve the uniformity of electric charge (and thus the uniformity of the electric field) across the substrate. Accordingly, the electrode layer is composed of a conductive material and is used to distribute the electric charge across the substrate.

In an example, the electrode layer forms a grid (also referred to herein as an electrode grid) having a plurality of apertures therein. For example, the electrode grid can be in the form of a square grid of connected vertical and horizontal lines of conductive material forming square apertures as shown in FIG. 10. In other examples, other types of grids and shapes of apertures may be used including circular, rectangular, or other shapes. In an example, the electrode layer can include a thin-film conducting layer composed of, for example, a single metal, metal alloy, stack of metal layers, conducting polymers, or the like. In an example, the electrode layer is composed of gold. As mentioned above, the electrode layer can be used to equalize conductivity across the substrate.

In an example, the electrode layer can be formed on the substrate by placing a layer of conductive material on the substrate (e.g., on top of the conductive material) and patterning the layer of conductive material using standard photo-lithography and deposition lithography. In some examples, the electrode layer can be disposed between the conductive material portion of the substrate and the base material portion of the substrate by, for example, forming the electrode layer on a base material of the substrate and then placing another layer of conductive material over the electrode layer.

In an example, the microwell array includes a walled portion. The walled portion can include a plurality of interconnected walls forming a plurality of apertures between the walls. Similar to the electrode layer, the walled portion may also form a grid structure, however, in some examples; the walled portion has a substantially larger height in order to contain material within the apertures of the walled structure. In an example, the walls of the walled structure can be substantially aligned with the conductive material of the electrode layer such that the apertures in the walled portion substantially align with gaps between the conductive material of the electrode layer. In an example where the electrode layer comprises a grid, the apertures of the walled structure can be substantially aligned with the apertures of the electrode grid. Accordingly, the walled portion can be formed or otherwise mounted on the substrate (e.g., over top of the electrode layer) and the apertures in the electrode grid can substantially align with the apertures of the walled portion to form a plurality of wells. Structurally, the walls of the walled portion form the sides of the wells and the substrate (e.g., the conductive material) can form the bottom of the wells. Accordingly, the wells are surrounded in the horizontal plane by the walls of the walled portion. Additionally, the electrode layer although typically being substantially shorter in height (e.g., perpendicular to the substrate) than the walled portion can be disposed between the wells in the horizontal plane. In an example, the electrode layer can substantially surround the wells in the horizontal plane (e.g., when the electrode layer is a grid).

In an example, the walled portion of the microwell array is created by spin coating a material (e.g., a photo-resist) on top of the substrate having the electrode layer thereon. The photo-resist can be patterned using photo-lithography to create the walled portion with wells (microwells) in between the walls. The photo-resist is developed and baked to create the microwells. In an example, the photo-resist material can include SU8, PDMS, thin (0.1-10 micron) and thick (10-500 micron) photoresists. In another example, the walled portion is created using molding techniques with thermo-setting plastics, followed by etching (dry or wet) to expose the substrate surface. In an example, the walled portion is fabricated in such a manner as to incorporate cell-repellent properties to minimize cell migration across the array by using chemically modified PDMS, SU8 or similar substances. In an example the walled portion is made cell-repellent by surface chemical modifications using materials such as Poly-ethylene glycol (PEG), Bovine Serum Albumin, or other non-specific binding blocking agent. In an example, the walled portion is made cell-repellent by the addition of a top layer made of a cell-repellent substance such as PEG, or modified PDMS.

In an example, the walled portion is created such that the walls of the walled portion overlay the conductive material of the electrode layer. In an example, the electrode layer is completely contained within the walls such that the conductive material of the electrode layer is not exposed in a well. A microwell array having having a conductive material on the bottom of a well is also referred to herein as a bottom electrode microwell array.

### Example 2

### Side Electrode Microwell Array

Example 2 begins with a substrate including a base material. In an example, the base material is non-opaque (e.g., transparent or translucent). The base material can be either flexible or rigid and is non-conductive. For example, the base material can include a glass including borosilicate, quartz, soda-lime, and porous or non-porous glass. In other examples, the based material can include a plastic (e.g., a polymer) including Poly-methyl-methacrylate (PMMA), Polystyrene, Polycarbonate, and Polypropylene. In another example, the base material is opaque and can be a silicon, ceramic and the like.

In an example, an electrode layer is formed on the substrate. In some examples, the electrode layer provides substantially uniform electric charge across the substrate. Accordingly, the electrode layer is composed of a conductive material and is used to distribute the electric charge across the substrate.

In an example, the electrode layer is composed of two or more portions that are not electrically coupled to one another. Accordingly, a first portion can be coupled to a positive charge and used as an anode and a second portion can be coupled to a negative charge and used as a cathode. In an example, the electrode layer is configured such that a well has at least two different portions of the electrode layer that adjacent to the well. For example, the electrode layer can comprise a plurality of parallel lines of conductive material. The parallel lines can alternate as cathodes and anodes and wells can be formed (as described below) between the parallel lines. Moreover, in some examples, different voltages can be applied to different portions across the substrate in order to create a uniform electric field in different wells across the substrate. In other examples, other shapes can be used.

In an example, the electrode layer can include a thin-film conducting layer composed of, for example, a single metal, metal alloy, stack of metal layers, conducting polymers, or the like. In an example, the electrode layer is composed of gold.

In an example, the electrode layer can be formed on the substrate by placing a layer of conductive material on the substrate and patterning the layer of conductive material using standard photo-lithography and deposition lithography. In some examples, the electrode layer can be disposed between the conductive material portion of the substrate and the base material portion of the substrate by, for example, forming the electrode layer on a base material of the substrate and then placing another layer of conductive material over the electrode layer.

In an example, the microwell array includes a walled portion. The walled portion can include a plurality of interconnected walls forming a plurality of apertures between the walls. The walled portion may form a grid structure and can typically have a height sufficient to contain material within the apertures of the walled structure. In an example, the walls of the walled structure can be substantially aligned with the conductive material of the electrode layer such that the apertures in the walled portion substantially align with gaps between the conductive material of the electrode layer. In an example where the electrode layer comprises a grid, the apertures of the walled structure can be substantially aligned with the apertures of the electrode grid. Accordingly, the walled portion can be formed or otherwise mounted on the substrate (e.g., over top of the electrode layer). Structurally, the walls of the walled portion form the sides of the wells and the substrate can form the bottom of the wells. Accordingly, the wells are surrounded in the horizontal plane by the walls of the walled portion. Additionally, the electrode layer although typically being substantially shorter in height (e.g., perpendicular to the substrate) than the walled portion can be disposed between the wells in the horizontal plane.

In an example, the walled portion of the microwell array is created by spin coating a material (e.g., a photo-resist) on top of the substrate having the electrode layer thereon. The photo-resist can be patterned using photo-lithography to create the walled portion with wells (microwells) in between the walls. The photo-resist is developed and baked to create the microwells. In an example, the photo-resist material can include SU8, PDMS, thin (0.1-10 micron) and thick (10-500 micron) photoresists. In another example, the walled portion is created using molding techniques with thermo-setting plastics, followed by etching (dry or wet) to expose the substrate surface. In an example, the walled portion is fabricated in such a manner as to incorporate cell-repellent properties to minimize cell migration across the array by using chemically modified PDMS, SU8 or similar substances. In an example the walled portion is made cell-repellent by surface chemical modifications using materials such as Poly-ethylene glycol (PEG), Bovine Serum Albumin, or other non-specific binding blocking agent. In an example, the walled portion is made cell-repellent by the addition of a top layer made of a cell-repellent substance such as PEG, or modified PDMS.

In an example, the walled portion is created such that the walls of the walled portion at least partially overlay the electrode layer. In an example, the electrode layer is not completely contained within the walls such that portions of the conductive material of the electrode layer are exposed in a well. Notably, two no-coupled portions of the electrode layer can be exposed in a well (e.g., on opposite sides of a well). Accordingly, the exposed portion of the first portion of the electrode layer can comprise an anode for the well and the exposed portion of the second portion of the electrode layer can comprise a cathode for the well. A microwell array having no conductive material on the bottom of a well (e.g., no conductive material in the substrate) and having an electrode layer which has two or more exposed non-coupled portions in a well is also referred to herein as a side electrode microwell array.

FIG. 12B illustrates examples of the electric field created during electroporation using the microwells discussed above with respect to FIG. 12A. As shown in the left column of FIG. 12B, in a bottom electrode microwell array, the electric current propagates through the electrode layer across the substrate. In a well, the electric current propagates from the electrode layer into the conductive material and the walled portion acts as an insulator. The electric current then goes into the electroporation buffer and onto an anode placed on top of the microwell array.

In the side electrode microwell array (right column of FIG. 12B) the electric current goes from a first exposed electrode portion on one side of the microwell to another exposed and opposite charged electrode portion on another side of the well through the electroporation buffer.

As mentioned above, in some examples the walled portion can be formed separate from the substrate and bonded to the substrate. For example, a walled structure having a plurality of apertures therein, for the wells, can be mounted to a first side of the substrate such that each aperture of the walled portion and the substrate form a microwell for containing a cell culture. The walled portion can be bonded to the substrate with a biocompatible adhesive. In another example, the walled portion can be bonded to the substrate using wafer bonding and stacking. In another example, the walled portion can be pressure or vacuum sealed onto the substrate. In an example, the walled portion can be composed of silicon, glass, metal, alloys, plastics, ceramics, polymers. In an example, the walled portion is permanently bonded to the substrate, however, in other examples, not permanently bonded to the rest of the substrate, such that it can be removed to facilitate analysis of the assay.

Example Microfabrication Protocols for Microwell Array Fabricated Directly onto Substrate

Indium Tin Oxide deposited polished slides were obtained (#CG-411N-S107, Delta Technologies, MN) and subsequently cleaned for 3 minutes with deionized water, methanol, and chloroform separately via water bath sonication. The slides were then air dried and dehydrated at 200 C over night in a vacuum oven (#1410, VWR, PA). Slides were first plasma treated for 5 minutes with ionized oxygen (PEIIB, Technics). Photoresist PMGI (Microchem, MA) was spin-coated at 500 rpm for 15 seconds and 130 acceleration and subsequently at 4500 rpm for 45 seconds and 1040 acceleration. The slides were then baked at 200 C for 5 minutes and allowed to cool for 10 minutes prior to the next spin-coat. A second layer photoresist (#10018357, Microchem, MA) was spin-coated at 500 rpm for 15 seconds and 130 acceleration and subsequently at 2500 rpm for 40 seconds and 260 acceleration. Slides were then pre-baked at 100 C for 20 minutes and subsequently allowed to cool for 15 minutes. Photo-lithography was then performed, whereby the slides were exposed for 20 seconds under UV radiation (MA6/BA6, Suss Microtech, Germany) at 7.5 W/cm². Slides were then developed for 3.5 minutes in MF319 developer (#10018042, ROHM+HAAS, PA), rinsed with deionized water, and finally nitrogen dried. Post baking of the slides was performed at 100 C for 10 minutes, and a second plasma etch was carried out before metallization. Metallization was performed in vacuum at 10⁻⁷ Torr (BJD 1800, Airco Temescal) whereby 20 nm of chromium at 2 Angstrom/second was deposited following 150 nm of gold at 2 Angstrom/second. Lift-off of the gold was performed overnight in resist stripper (Remover PG, Microchem, MA). Next day, the remaining gold was gently rubbed off, rinsed in ethanol, and nitrogen dried. Alternatively, metallization was performed via sputtering (Discovery 18, Denton Vacuum LLC, NJ) whereby 20 nm of chromium was deposited at 0.45 Angstrom/second following 150 nm of gold at 8.3 Angstrom/second in a vacuum at 2.8e⁻⁶ Torr. Lift off was performed overnight outside of the clean room submerged in Remover PG with gentle shaking. After metallization and lift-off, an initial plasma etch was carried out to clean the surface. Slides were spincoated with SU8-2050 (Microchem, MA) for 15 seconds at 500 rpm and 130 acceleration and subsequently for 40 seconds at 4000 rpm and 260 acceleration. A soft bake was then performed at 60 C for 2 minutes and then 100 C for 7 minutes. Photo-lithography was then performed, and the slides were exposed to UV radiation for 60 seconds (MA6/BA6, Suss Microtech, Germany). A post exposure bake was performed at 60 C for 3 minutes and then at 100 C for 8 minutes. Slides were allowed to cool for 15 minutes and then subsequently developed (#Y020100 4000L1PE, Microchem, MA) for 6 minutes. After a 1 minute isopropanol wash, the slides were nitrogen dried and hard baked at 160 C for 2-3 hours.

### Sample Protocol for Chemical Modification of the Substrate to Provide a Positive Electrostatic Charge on the Surface

After the microfabricated slides were baked, a plasma etch was performed for 6 minutes (Plasma Prep II, SPI Supplies, PA) at 150-200 mTorr with ionized oxygen. The slides were then immediately immersed in 2.5% of 3-Aminopropyltriethoxysilane 99% (#440140, Sigma Aldrich) in room temperature toluene for 6 hours. Following silanization, the slides were washed with toluene twice, ethanol once, and finally dried via nitrogen.

### Sample Electroporation Protocol for Microwell Array Fabricated Directly Onto Substrate

Electroporation experiments were carried out in a modified setup using a square wave electroporator (ECM 830 Electroporation System, BTX, MA). The effective electroporation distance was set at 1 mm, and the buffer used was room temperature Opti-MEM (#31985, Invitrogen, CA).

### Section B—Microwell Preparation

Referring now to section B of FIG. 2, after creation of the microwell array, the microwells are prepared for introduction of cells and exogenous molecules, and electroporation of cells. In some examples, the bonded microwell arrays are sterilized, washed with PBS and then soaked in a cell adhesion substrate. In one example, fibronectin (Sigma) is employed to increase cell adhesion within the microwells. In other examples, other cell adhesion substrates are employed. The microwells are then washed in PBS to remove the unbound cell adhesion substrate and placed in a tissue culture dish.

In certain embodiments, the surface of the base portion of the substrate is modified by the addition of natural or synthetic polymers, peptides, proteins, lipids, or the like. In some embodiments, the surface of the base portion of the substrate is chemically modified to facilitate and control the binding and release of the exogenous molecules. For example, a positive electrostatic charge can be provided on the surface of the microwell substrate to aid in binding and release of nucleic acids. See FIG. 11C. When the cells are introduced into the microwells, the exogenous molecules are released from the substrate by diffusion prior to and during electroporation, as the electrostatic charge is reversed.

In some embodiments, the substrate is plasma etched and silanized to generate a positive electrostatic charge. In some embodiments, the substrate is silanized using amino-propyl-triethoxy silane (APTES) to modulate the electrostatic charge at the surface towards a net positive charge. In other embodiments, the base portion is chemically modified to modulate the electrostatic charge at the surface towards a net negative charge.

In certain embodiments, the base portion is chemically modified to make the surface more hydrophobic or more hydrophilic. In other embodiments, the base portion is itself chemically modified to facilitate intelligent release (i.e. controlled by a timely external input) of the exogenous molecule. Such a timely external input can include, but not be limited to, a change in electric current, a change or presence in light of any wavelength, a variation in temperature, or a chemical agent of any kind (including reducing and oxidizing factors).

In certain embodiments, the surface of the conductive material is modified by the addition of natural or synthetic polymers, peptides, proteins, lipids, or the like. In some embodiments, the surface of the conductive material is chemically modified to facilitate and control the binding and release of the exogenous molecules. For example, a positive electrostatic charge can be provided on the surface of the conductive material to aid in binding and release of nucleic acids.

In some embodiments, the conductive material is silanized to generate a positive electrostatic charge. In some embodiments, the conductive material is silanized using amino-propyl-triethoxy silane (APTES) to modulate the electrostatic charge at the surface towards a net positive charge. In other embodiments, the conductive material is chemically modified to modulate the electrostatic charge at the surface towards a net negative charge.

In certain embodiments, the conductive material is chemically modified to make the surface more hydrophobic or more hydrophilic. In other embodiments, the conductive material is itself chemically modified to facilitate intelligent release (i.e. controlled by a timely external input) of the exogenous molecule. Such a timely external input can include, but not be limited to, a change in electric current, a change or presence in light of any wavelength, a variation in temperature, or a chemical agent of any kind (including reducing and oxidizing factors).

Section C—Introducing Cells and Exogenous Molecules into Microwells

After the microwells are prepared, cells and exogenous molecules are introduced into the microwells. In some embodiments, the cells are added to the microwells before the exogenous molecules. In other embodiments, the exogenous molecules are added to the microwells before the cells. In still other embodiments, the exogenous molecules and cells are added to the microwells at the same time.

After the microwells are prepared, exogenous molecules are added to the microwells. In some embodiments, exogenous molecules are added using a liquid handler. In other embodiments, exogenous molecules are added using a pin-based contact microarrayer using an iterative method of alignment. In still further embodiments, exogenous molecules are added using a non-contact microarrayer.

In certain embodiments, the exogenous molecule is mixed with a controlled release agent (such as a biodegradable, chemically degradable, photocleavable, naturally dissolving, or thermally denaturing material) before addition to the microwell. This facilitates the controlled release of the molecule in the microwell prior to electroporation.

After the microwells are prepared, cells are seeded by placement of the substrate and cells in a culture dish. In some examples, the culture dish is a 10 cm dish. Cells are then incubated at the appropriate temperature for a brief period of time, such as for 1 hr post-seeding. The arrays are then washed to remove unbound cells. Fresh media is then added. Cells inside the microwells experience minimal flow stress and remain attached during this step. In one example, cells are then incubated for 24 hours. In a preferred method the cells are seeded using a microfluidic system to facilitate uniform cell seeding. In some embodiments the cells are seeded using a liquid dispenser in individual microwells or onto the entire microwell array to facilitate uniform cell seeding. In some embodiments the microwell array is inverted after cell attachment to remove cells not attached within the microwells. In one embodiment a physical force is used to increase speed of cell seeding in the microwells. In one example, centrifugal force is used to increase speed of cell seeding. In another example, electrical fields (creating effects such as dielectrophoresis) are used to increase speed of cell seeding. In another example, magnetic force is used to increase speed of cell seeding of cells labeled with magnetic particles. In another example, hydrodynamic pressures are used to increase the speed of cell seeding. In one embodiment, height of the seeding chamber is lowered to increase the speed of cell seeding.

Cells contemplated for use in the microwells and methods of the invention described herein include prokaryotic cells, eukaryotic cells, single-celled organisms, bacterial cells, yeast cells, insect cells including Drosophila cells, murine cells, and human cells. In some embodiments, cells contemplated for use in the microwells and methods described herein include but are not limited to a tissue-derived cell, a patient-derived cell, a tumor-derived cell, primary cells and immortal cell lines. In some embodiments, cells that are amenable to electroporation are employed in the methods described herein. In other embodiments, cells that are not traditionally amenable to electroporation are employed in the methods described herein.

Exogenous molecules can also be introduced into the microwells before electroporation. In some examples, exogenous molecules are introduced in solution. In other examples, the exogenous molecules are spotted on the microwells before the cells are introduced. Some embodiments include the use of surface chemistry to bind and release the exogenous molecules at desired time points, such as shown in FIG. 11C. The use of positive and negative charges for electrostatic assembly and disassembly of exogenous molecules is contemplated.

The exogenous molecules that are transfected into the cells described herein by electroporation using the described apparatus, which may comprise a unitary molecule or a plurality of different molecules, may include, but are not intended to be limited to, amino acids, bioactive molecules, natural or synthetic polypeptides, peptide aptamers, proteins, antibodies (or fractions thereof), glycoproteins, enzymes, nucleic acids (natural or synthetic, including analogs such as morpholino oligomers), oligonucleotides, polynucleotides, RNA (including but not limited to siRNA, ncRNA, miRNA, RNA of chemically-modified backbone (“locked RNA”), dsRNA, tRNA, ribozyme, an RNA aptamer, a Piwi-interacting RNA (piRNA), or other RNA species), DNA (natural or synthetic), competent DNA, plasmid DNA, shDNA, single or double stranded DNA oligos, antisense DNA, chromosomes, viruses including natural or synthetic viruses, virions, or virus protein assembly, including a retrovirus, an adenovirus, a DNA virus, a vaccinia virus, a cowpea mosaic virus, or any other virus or virion, a bacteriophage, drugs such as natural or synthetic small molecules including natural or unnatural lipids, and other charged organic or inorganic molecules that may or may not have a localized charge region. In some embodiments, the exogenous molecule is a nanoparticle (e.g., Quantum dot), a Lipid Nanoparticle (LNP), a virion, a protein assembly, or the like. In preferred examples, the exogenous molecules are nucleic acids. In some embodiments, the surface chemistry of the microwells is modified for binding and intelligent release of the molecules.

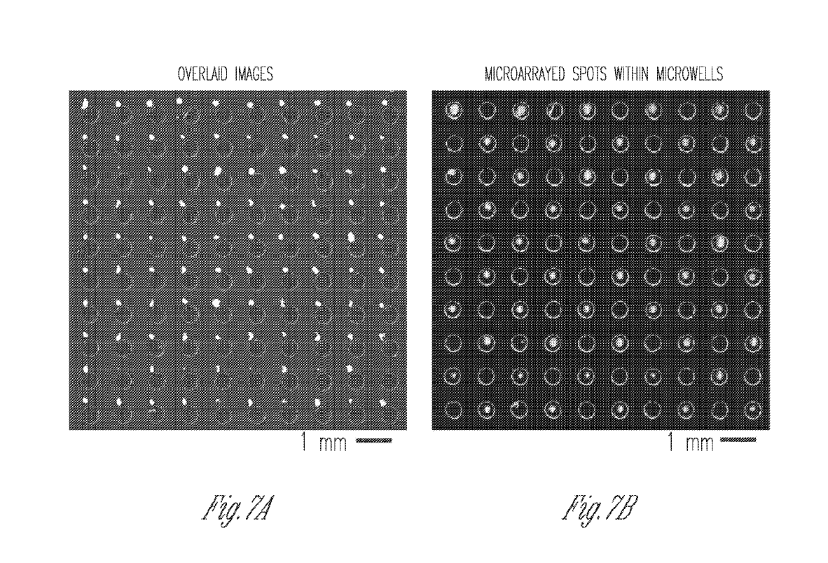

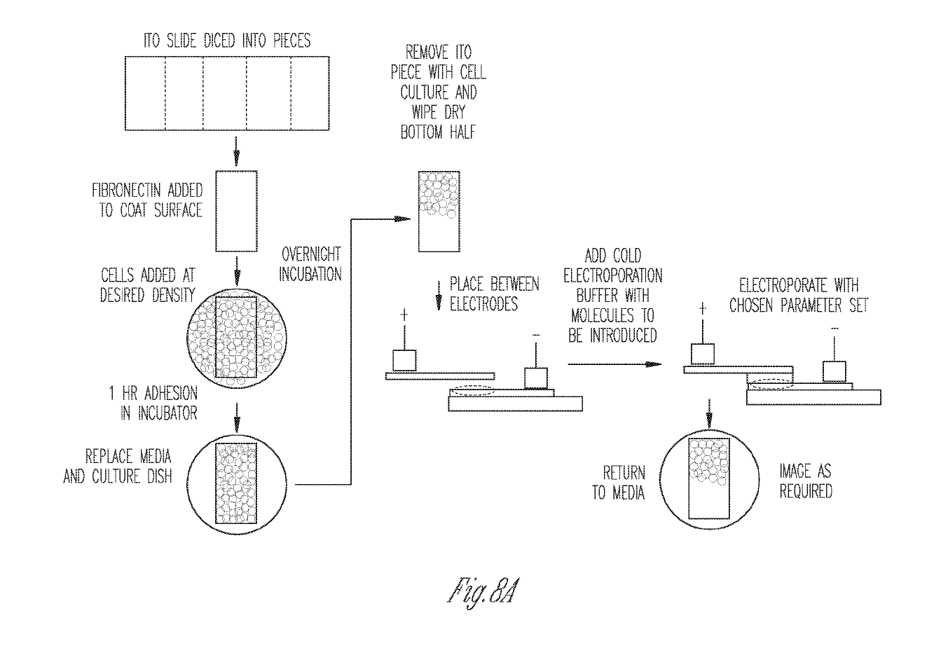

When introducing thousands of different exogenous molecules, loading large libraries of molecules onto the microstructure-bearing substrate requires precision. In some examples, the microarrayer is aligned so that exogenous molecules are spotted precisely within individual microwells. In some examples, a CCD camera is employed in conjunction with the microarraying head to image the substrate and locate the features prior to printing. In a preferred example, an iterative method is employed to align microwells with the microarrayer to insure accurate spotting on the experimental substrate. First, a blank microscope slide is spotted with printing buffer using the same spotting parameters (inter-spot distance and array size) to be used on the final microwell array slide. This blank spotted slide and the microwell array-containing slide are then imaged independently in a slide scanner. Next, the two images are overlaid using image analysis software to determine X-Y alignment errors (see FIG. 7A). Re-calibration of the microarrayer with these errors enables spotting to take place precisely within the microwell array. Using this approach, it is possible to consistently microarray within the center of microwells (see FIG. 7B).

### Section D—Electroporation of the Microwell Array

In one embodiment of electroporation, cells are seeded in microwells and electroporated within seconds or minutes. In another embodiment, cells seeded in microwells are electroporated after one day of incubation. In some embodiments cells are cultured for several days in microwells (such as required for differentiation or growth) before electroporation. In another embodiment, cells in microwells are electroporated more than once over the course of experimentation. In one embodiment electroporation buffer is replaced with media after several minutes to an hour, to allow cells to re-attach to the microwell.

In one embodiment electroporation is conferred with unipolar or bipolar square wave pulses of known voltage strength, pulse width, frequency and number. In one embodiment, electroporation is conferred with radio frequency pulses. In one embodiment electroporation is conferred using exponentially decaying pulses. In one embodiment electroporation is conferred using complex waveforms comprising of multiple frequencies. In one embodiment electroporation is conducted at room temperature. In one embodiment electroporation is conferred at low temperatures (such as by keeping the buffer on ice).

As shown at D of FIG. 2 and at C of FIG. 11, after seeding of the cells, the microwell array is electroporated. In alternate embodiments, the polarity of the electrodes can be changed. In some examples, prior to electroporation, the left and right flanks of the ITO slides are wiped dry to create an electrolyte free area for cathode placement, keeping the microwell array wet with residual media. In some examples, a hydrophobic barrier is put on both the left and right flanks of the array to restrict the electroporation buffer to the top of the microwell array. In some examples, a hydrophobic barrier pen is used to draw the hydrophobic barriers on the microwell array.

To electroporate the microwell array a first electrode is located above the microwell array which is located on the conductive material surface (e.g. ITO) of the substrate. The conductive material surface of the substrate acts as a second electrode to complete the circuit from the first electrode above the microwell array. During electroporation current conducted between the two electrodes propagates through the microwells in the microwell array. In one example, the first electrode comprises a stainless steel anode and is placed at a 1 mm space from the conductive material surface using glass spacers. The second electrode is created by coupling a stainless steel cathode to the conductive material surface of the substrate. In order to aid in coupling the cathode to the conductive material substrate, a contact strip of copper tape, such as that manufactured by 3M Inc., MN, is placed on the conductive material surface of the substrate and the cathode is coupled to the contact strip.

In one example, the cathode is coupled to a single side of the substrate. In another example, referred to herein as a double cathode scheme, the cathode is coupled to the substrate at two locations, one location at each side of the substrate as shown in D of FIG. 2. In yet other examples, more than two locations are used to couple the cathode to the conductive material surface of the substrate. Coupling the cathode to more than one location of the substrate reduce voltage drops across the substrate and creates a more uniform electric field across the substrate. Uniform electroporation efficiency across the microwell array allows for highly parallel electroporation on a single substrate. In still other examples, patterns of cathode contacts on the substrate are used to further reduce voltage drop and create a more uniform electric field. In another example, the cathode is coupled to the substrate in a single location and the anode is set at a slight angle (˜1 degree) relative to the substrate such that one end of the anode is closer to the substrate than the other end of the anode. Notably, the portion of the anode that is closer to the substrate corresponds to the portion of the substrate that is farthest from the point at which the cathode is coupled to the substrate. Thus, the closeness of the anode counteracts the resistance of the substrate to reduce variation in the electric field across the substrate. In some embodiments, the anode is modified structurally (such as by adding a curvature to the anode) to match the resistance at each point of the substrate so that the electric field is constant throughout the experimental area.

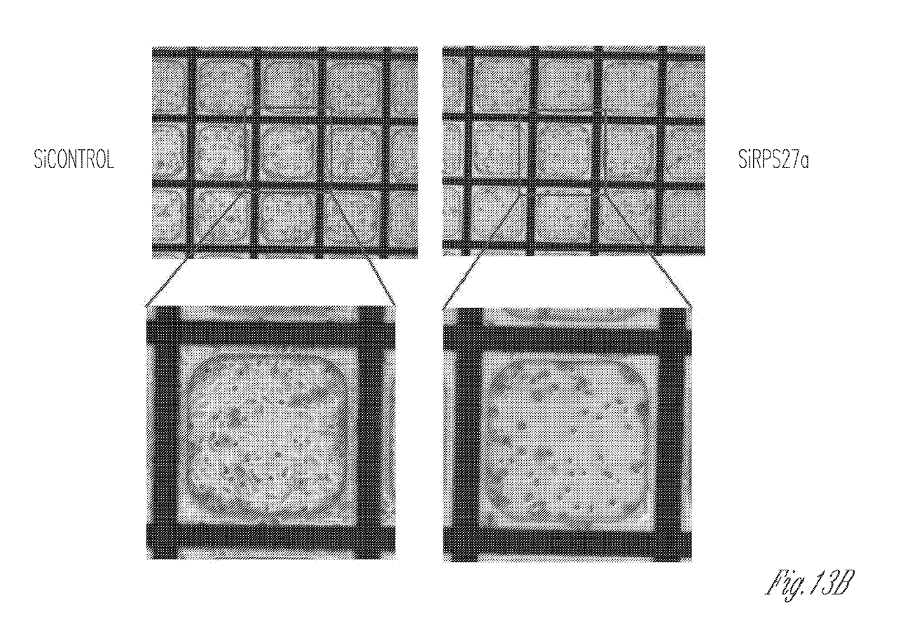

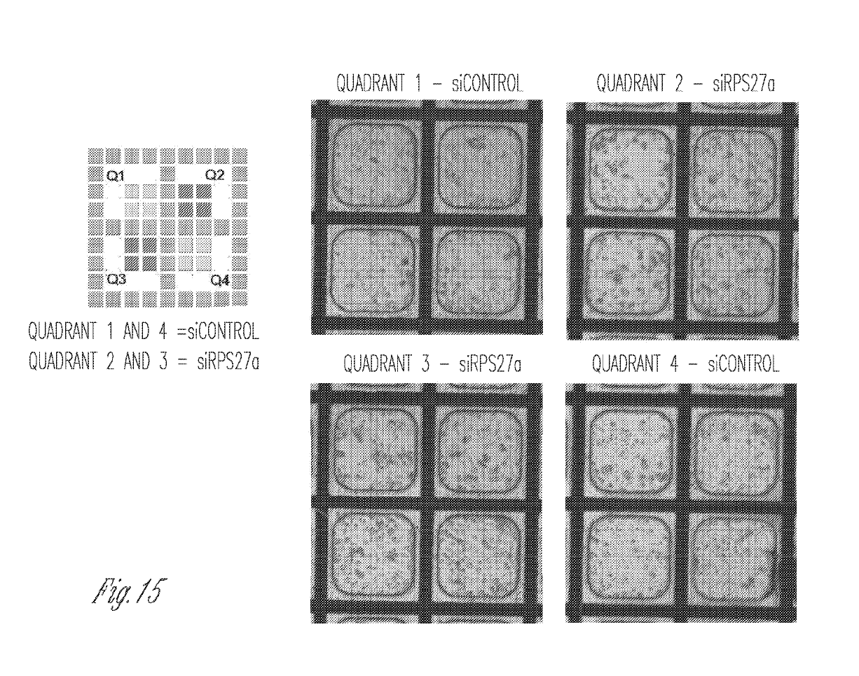

Once the electrodes are in place for electroporation, an electroporation buffer containing the molecules to be transfected into the cells is added to the space between the anode and the microwell array. For example, in one embodiment, ice cold propidium iodide at 40 μg ml⁻¹ is used as the exogenous molecule (see FIG. 13A). In other embodiments, other exogenous molecules are used, such as Alexa-488 fluor conjugated siRNA, or RPS27a siRNA (see FIGS. 13A and B and FIG. 15), or a plasmid encoding GFP (see FIG. 13C).

In some examples, electroporation is then simultaneously conferred in all microwells with the exogenous molecule in solution. In one example, electroporation is conferred using electroporation parameters of 1 pulse having a length of 1 ms and a causing an electric field strength of 500 V cm⁻¹. In other examples, other electroporation parameters are used. For example, higher or lower electric field strength may be generated and/or other numbers and/or lengths of pulses may be used. More detail regarding electroporation and electroporation parameters is provided below with respect to FIG. 10A.

After electroporation, the microwell array is placed back in media and incubated for an additional time period.

FIGS. 11A-C illustrates another example of high-throughput screening using a microwell array. FIG. 11A illustrates a top view of microwell arrays as well as an illustration of an experimental flow using a microwell array. Libraries of molecules are mapped/loaded from stock plates into segregated microwells on a microwell array. Cells are seeded into the microwells and the loaded molecules are electroporated. The microwell arrays with cells are incubated in media, assayed, imaged and analyzed to screen for hits.

FIG. 11B illustrates a cross-sectional view of a microwell array. The microwell array is fabricated and prepared for loading libraries of molecules. Liquid handling equipment (such as pin-based microarrayers or piezo-electric dispensers) is used to load the molecules in addressable microwells. Cells are then seeded within the microwells and electroporation is carried out. Cells within specific microwells are electroporated with only the existing molecules loaded in that microwell.

FIG. 11C illustrates a schematic showing two different methods to bind and release exogenous molecules in the microwells during cell seeding and prior to their electroporation. Left column: surface charges (positive in this case) can be used to bind the exogenous molecules electrostatically. Upon cell seeding, the molecules begin to slowly diffuse from the surface (a process that accelerates as the electrostatic charge is reversed during electroporation) and are henceforth electroporated into the overlaying cells. Right column: exogenous molecules are loaded with biodegradable/dissoluble release agents. Upon cell seeding the release agent degrades/dissolves, resulting in high concentration of free molecules inside the microwell, which are then electroporated

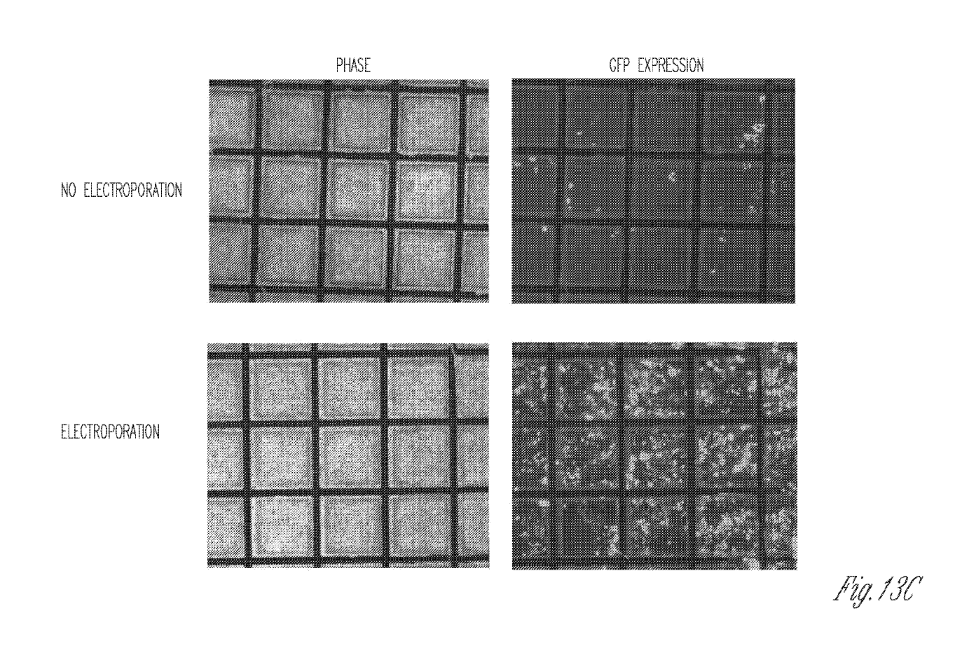

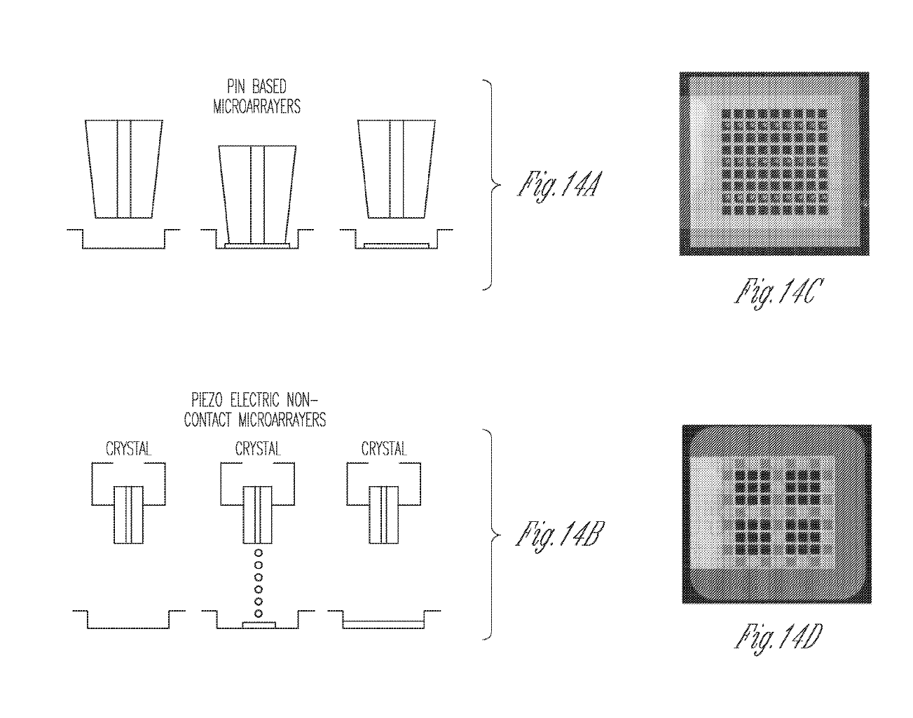

FIG. 14 illustrates examples of loading of microwell arrays with exogenous molecules. Two different example equipment types are shown to accomplish precisely aligned molecule loading in microwells. Section A of FIG. 14 illustrates a schematic of molecule loading using a contact pin-based microarrayer equipment. The tip delivers the solution containing the molecule by touching the bottom surface of the microwell. Section B of FIG. 14 illustrates a schematic of molecule loading using a non-contact piezo-electric dispenser equipment. The head dispenses sufficient solution containing the exogenous molecules to load the microwell. Sections C and D illustrate examples of aligned loading fluorescent-conjugated siRNA molecules in microwell arrays using either the contact pin microarrayer or piezo-electric dispenser (dispense volume per microwell was 10 mL) respectively. Microwells are 400 micron square dimensions and 500 micron separated.

### Electroporation Parameter Selection

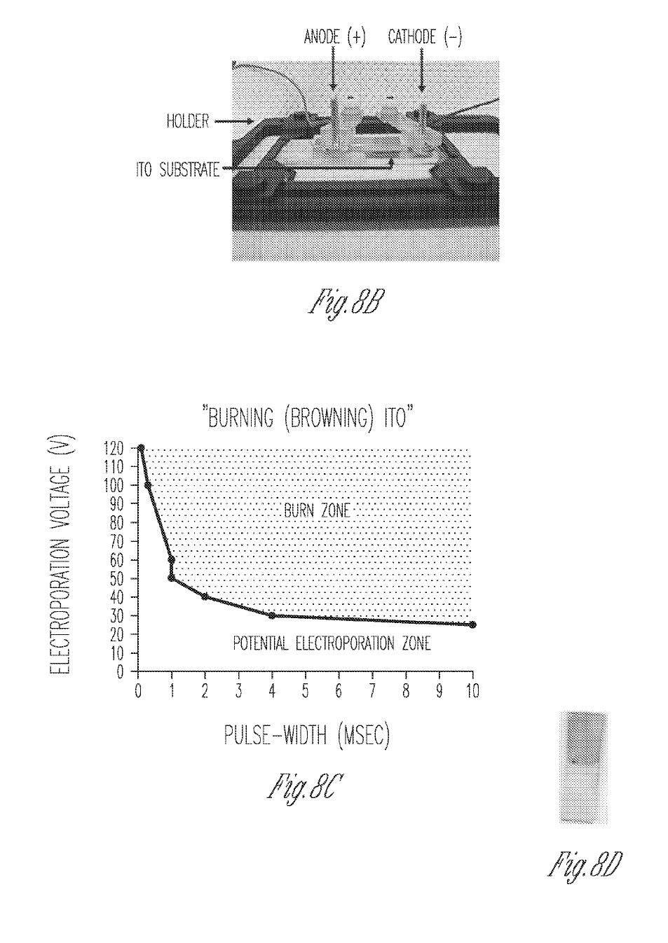

Referring now to FIG. 8A, one example of a method according to the invention to determine electroporation parameters for parallel electroporation in a microwell array is illustrated. Electroporation parameters are selected to produce a desired electric field through the microwell array. In the example shown in FIG. 8A, several criteria are used to determine the desired electric field including: the ability to introduce exogenous molecules from solution into cells growing on the ITO with high efficiency and minimal loss of cell viability and variations across the electric field. In other examples, other criteria are used to determine the desired electric field.

In the example shown in FIG. 8A, desired electroporation parameters are determined for HEK 293T cells. To determine the desired electroporation parameters, a variety of electroporation parameters (differential voltage, pulse-width and number of pulses) are tried. In this example, the electroporation parameters are tested with propidium iodide and HEK 293T cells cultured on the ITO coated glass substrates.

Electroporation parameters are selected in order to reduce burning of the conductive surface of the substrate. FIG. 8C is a graph showing an example of voltages and pulse-widths that result in burning of the conductive surface of the substrate. As shown the voltages and pulse-widths above the line results in burning of the conductive surface of the substrate. Voltages and pulse-widths below the line are preferable for electroporation.

Another criterion for selection of electroporation parameters is the resultant electric field strength, uniformity, and resulting transfection variability. The number and location of the cathode coupling locations also affects the electric field strength and uniformity.

### Section E—Image Acquisition and Analysis

Referring back to FIG. 2, section E shows image analysis after electroporation the microwells. The image analysis conducts phenotypic evaluation of the electroporated microwell array. First at least one image of the electroporated microwell array is captured. Once the image(s) are obtained, the spatial location of each cellular culture of interest is identified on the substrate during image analysis and phenotype deconvolution. To identify the spatial location of each cellular culture, the walls of the microwell array are identified and used to define the microwells between the walls. In one example, image analysis is conducted by a processing unit executing instructions stored on a computer readable medium. The processing unit is coupled to the computer readable medium executes the instructions to perform the acts of image analysis. In some examples, the image of the real world microwell array is analyzed by processing unit, and graphical or other illustrative results are displayed on a display device communicatively coupled to the processing unit.

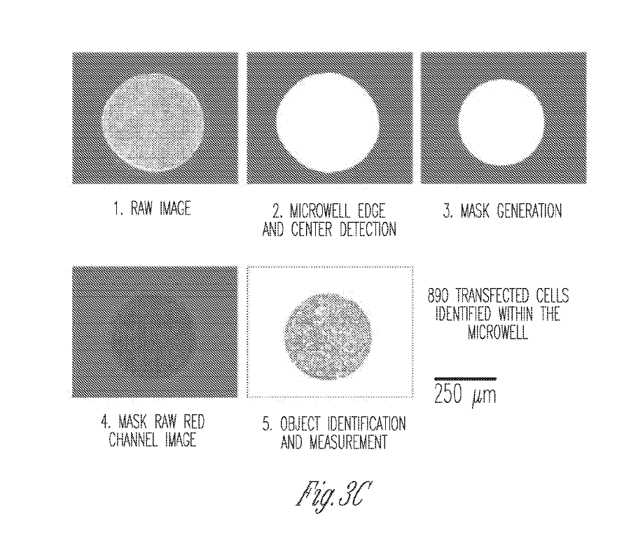

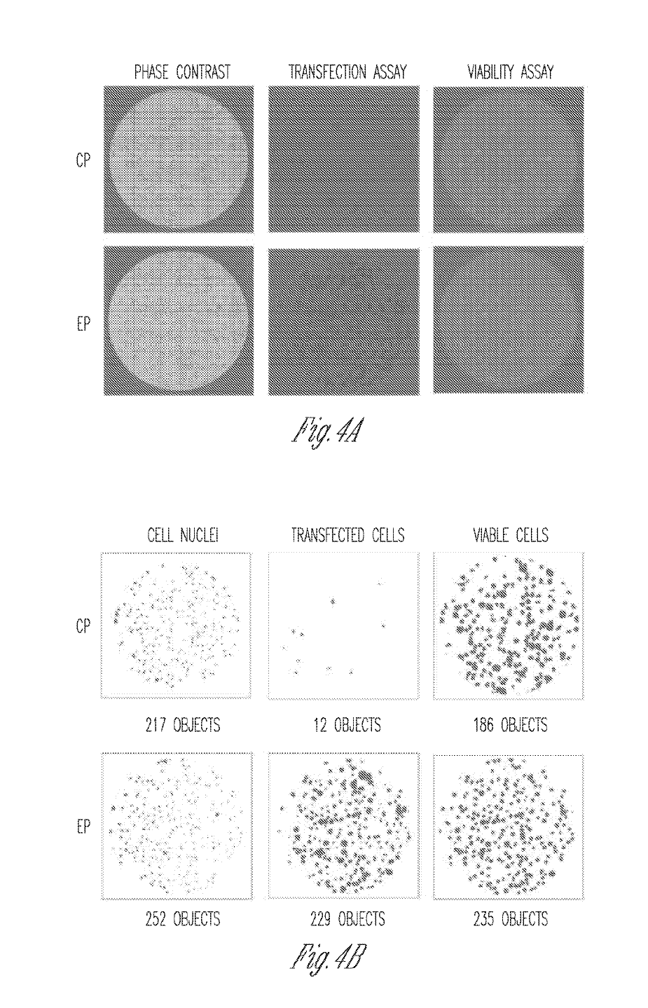

As shown in FIG. 3C, using the walls of the microwell array as a physical marker, the spatial location of the microscale cultures can be identified. In one example, the edges of the microwells are identified from the image, which in turn is used to determine the center of the microscale culture position. The processing unit then crops out the microscale culture removing the unwanted microwell edges. With subsequent modules for thresholding, segmentation, and object identification, a variety of phenotypic analysis can be accomplished (in this example simply identifying 890 electroporated cells within a microwell). The presence of the microwells dramatically enhances the ability to precisely identify the micro scale cellular cultures on the substrate, thus increasing the accuracy of phenotypic annotation.

In one example, the following cameras and software may be used for image analysis and/or to perform other acts throughout the method shown in FIG. 2. Cellular imaging of microscale cultures within individual microwells is carried out on a Nikon eclipse TE-2000U inverted fluorescence microscope with a cooled ccd camera (CoolSnap fx, Photometrics, AZ). The cellular images are then analyzed using NIH ImageJ (http://rsb.info.nih.gov/ij) for analysis of transfection, GFP expression and viability. High-resolution imaging of the entire 484-microwell array is conducted on a ProScanArray HT confocal laser slide scanner (Perkin Elmer, MA) and the images were analyzed with ImaGene software (BioDiscovery, CA) to determine individual microwell fluorescence intensities. In other examples, however, other cameras and/or microscopes are used.

The presently disclosed subject matter may be illustrated by the following non-limiting example.

### Example

### Highly Parallel Introduction of Nucleic Acids into Mammalian Cells Grown in Microwell Arrays

### Materials and Methods

### Optimization of Electroporation Parameters for HEK 293T Cells

Optimization was conducted on diced Indium-Tin Oxide (ITO) coated glass (unpolished, surface resistivity 4-8 Ωsq⁻¹, Delta Technologies, MN) pieces as shown in FIG. 8A, on a custom-built electroporation setup (FIG. 8B). Briefly, pieces 1 cm×2.5 cm were diced from single microscope slides, rinsed in de-ionized water and dried under a nitrogen stream. Thereafter, 100 μl of 10 μg m⁻¹ fibronectin from human plasma (Sigma) was pipetted on the top half of the piece and allowed to coat for 2 hrs. After aspiration and washing with PBS, 2−3×10⁴ HEK293T cells in 100 μl of media (DMEM, 10% FBS and antibiotics) were added to the same spot. The cells were allowed to adhere to the spot for 1 hr in the incubator at 37° C. and 5% CO₂, prior to washing with PBS and flooding with media. After 24 hrs of incubation, media was aspirated and the cultures were immediately placed in the electroporation setup. Alternatively, 2×10⁵ cells were plated within each well of a 6-well dish on top of the pieces. Prior to electroporation, the bottom half of the pieces were wiped to create an electrolyte free area for cathode placement.

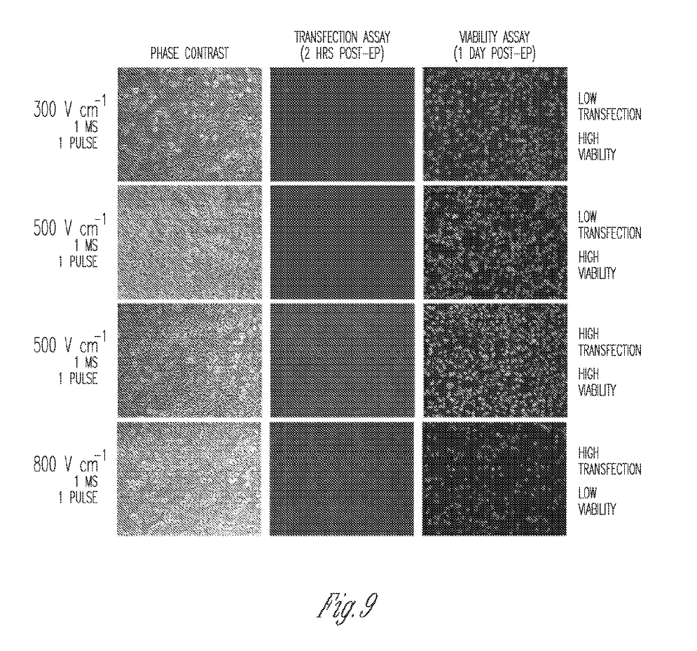

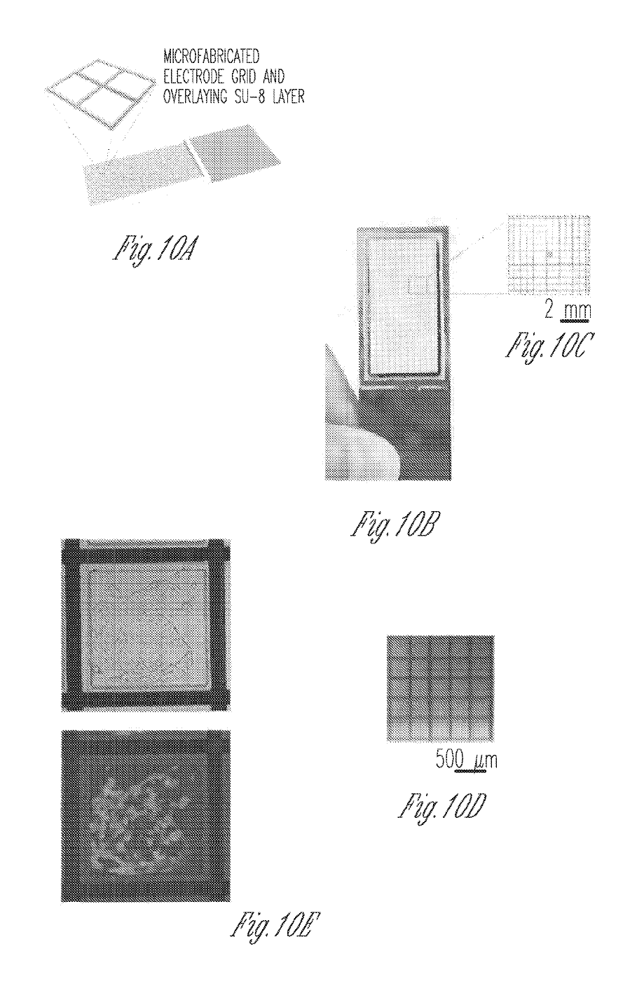

A stainless steel anode was placed on top of the ITO piece at a spacing of 1 mm and ice cold electroporation buffer (5.5 mM D-Glucose, 137 mM NaCl, 5.4 mM KCl, 0.44 mM KH₂PO₄, 4.1 mM NaHCO₃, 20 mM HEPES) was added between the two electrodes (the ITO conductive substrate being the cathode) of a BTX square-wave pulse electroporator (ECM830, Genetronics, CA). Several combinations of electroporation parameters, such as electric field (50 V cm⁻¹ to 800 V cm⁻¹), pulse-width (0.1 ms to 100 ms), and number of pulses (1-8) were applied to the electrodes. Additional discussion of the strategy for optimization of electroporation conditions for different cell lines can be found in the legend accompanying FIG. 9. Propidium Iodide (40 μg/ml in electroporation buffer) was electroporated with each parameter set to determine transfection efficiency and cells were imaged 2 hr post-electroporation. Electroporated cells were incubated with a lentivirus encoding green fluorescent protein GFP (viral-GFP particles) for 24 hr to assess cell viability. Alternatively, cell viability was evaluated using 1 μg ml⁻¹ of Calcein AM (Invitrogen). Alexa-488 fluor conjugated siRNA (Qiagen) was used at a concentration of 5 μM in electroporation buffer as a test for successful electroporation of siRNA molecules. Control (no electroporation) parameters in each case were 100 V cm⁻¹, 1 ms and 1 pulse.

Parallel Electroporation within Microwell Arrays

A 484-microwell array was created by essentially sticking a laser cut coverlay (FlexTac BGA Rework Stencil 22×22 array, thickness 100 μm, CircuitMedic, MA) to the center of a conductive ITO coated microscope slide using the pre-coated adhesive provided on the backside. Microwells were 500 μm in diameter and separated at 1 mm inter-well distance. The bonded microwell arrays were sterilized, washed with PBS and then soaked in 1 μg ml⁻¹ fibronectin (Sigma) to increase cell adhesion within the microwells. The microwells were then washed in PBS to remove the unbound fibronectin and placed in a 10 cm tissue culture dish. Thereafter, 7.5×10⁶ HEK 293T cells were seeded into the tissue culture dish containing the microwell array and placed in the incubator. 1 hr post-seeding the arrays were washed to remove unbound cells and fresh media was added.

The 484-microwell array ITO slides containing the microscale cultures were removed from the incubator after 24 hr. The left and right flanks of the ITO slides were wiped dry, keeping the microwell array wet with residual media. A hydrophobic barrier pen (Ted Pella, CA) was used to draw hydrophobic barriers on both the left and right flanks of the microwell array to restrict the electroporation buffer to the top of the microwell array. A stainless steel anode was placed at 1 mm space from the ITO surface using glass spacers. A stainless steel electrode provided contact to one end of the ITO slide to be used as a cathode. In the case of using a double cathode scheme, a conductive copper tape (3M Inc., MN) electrically shorted both ends of the ITO slide. Ice cold electroporation buffer containing the molecules to transfected (propidium iodide at 40 μg ml⁻¹, Alexa-488 fluor conjugated siRNA at 5 μM or a plasmid encoding GFP at 300 μg ml⁻¹) were added to the space between the anode and the microwell array. Electroporation was simultaneously conferred in all 484-microwells using an optimal parameter set as determined during optimization experiments (500 V cm⁻¹, 1 ms pulse-width and 1 pulse). Thereafter, the microwell array was placed back in media and incubated for an additional 2 hr, with subsequent staining for viability using Calcein AM (Invitrogen, CA). Thereafter, the microculture array was fixed with 10% formalin (Sigma) and stored at 4° C. In the case of plasmid electroporation, GFP expression was assessed at 24 hr post-electroporation.

### Electroporation of Primary Mouse Macrophages in Microwell Arrays

Primary mouse macrophages were isolated from 2- to 3-month-old male C57BL/6 mice as described¹⁶. Thioglycollate-elicited peritoneal macrophages were plated on top of microwell ITO substrates in six-well plates at density of 1×10⁶ cells per well. This seeding density achieved 30-50% confluence/microwell at 24 hr. Electroporation of propidium iodide was conducted as described above for ITO pieces. Parameters tested included electric field, pulse-width, and number of pulses. 2 hr post-electroporation, a live assay was conducted using Calcein AM (Invitrogen, CA) to determine remaining viability and nuclei stained with Hoechst (Invitrogen, CA) to determine the total number of cells within the microwells.

### Finite Element Modeling and Analysis of Electric Field

To study the variations of electric field on the microwell array region on an ITO slide, finite element modeling and analysis was conducted in FEMLAB (Comsol, CA) using the conductive DC module. The ITO surface was modeled with a thickness of 200 nm and material properties of conductivity 3.75e⁶ S m⁻¹ and surface resistivity of 4 Ωsq⁻¹. The anode was modeled as a positive contact (voltage 50 V) at a distance of 1 mm from the surface of the ITO, which served as the cathode (voltage 0 V). The intermediate region was modeled as a conductive media of conductivity 1.6 S m⁻¹, equivalent to the measured conductivity of the electroporation buffer. After meshing and solving, the electric field intensities parallel to the z-axis (normal to the ITO surface) were plotted for analysis.

### Cellular Imaging and Analysis

Cellular imaging of microscale cultures within individual microwells was carried out on a Nikon eclipse TE-2000U inverted fluorescence microscope with a cooled ccd camera (CoolSnap fx, Photometrics, AZ). Cellular images were analyzed in NIH ImageJ (http://rsb.info.nih.gov/ij) for analysis of transfection, GFP expression and viability. High-resolution imaging of the entire 484-microwell array was conducted on a ProScanArray HT confocal laser slide scanner (Perkin Elmer, MA) and the images were analyzed with ImaGene software (BioDiscovery, CA) to determine individual microwell fluorescence intensities.

Microarraying within Microwell Arrays

Microarraying was carried out on a Biorobotics MicroGrid II microarrayer (Genomic Solutions, IL). To align the microwell array with the microarraying pins, a regular glass microscope slide was spotted with Alexa 488 fluor conjugated siRNA (1 μM) and imaged on the ProScanArray HT confocal laser slide scanner (Perkin Elmer, MA). Similarly, the microwell array to be spotted within was imaged on the scanner at the identical settings. The two images were then overlaid in ImaGene software (BioDiscovery, CA) and the X and Y offset of the microwells from the microarrayed spots was determined. The microarrayer was then recalibrated with the offsets. Upon confirmation of precise alignment of the microwell array and the microarrayed spots in the imaging software, Alexa Fluor 488-labeled siRNA was microarrayed directly within the microwells. A stealth pin SMP10B (Arrayit, CA) with a spotting diameter of 365 μm was used for the arraying.

### Results

### Optimization of Electroporation on ITO

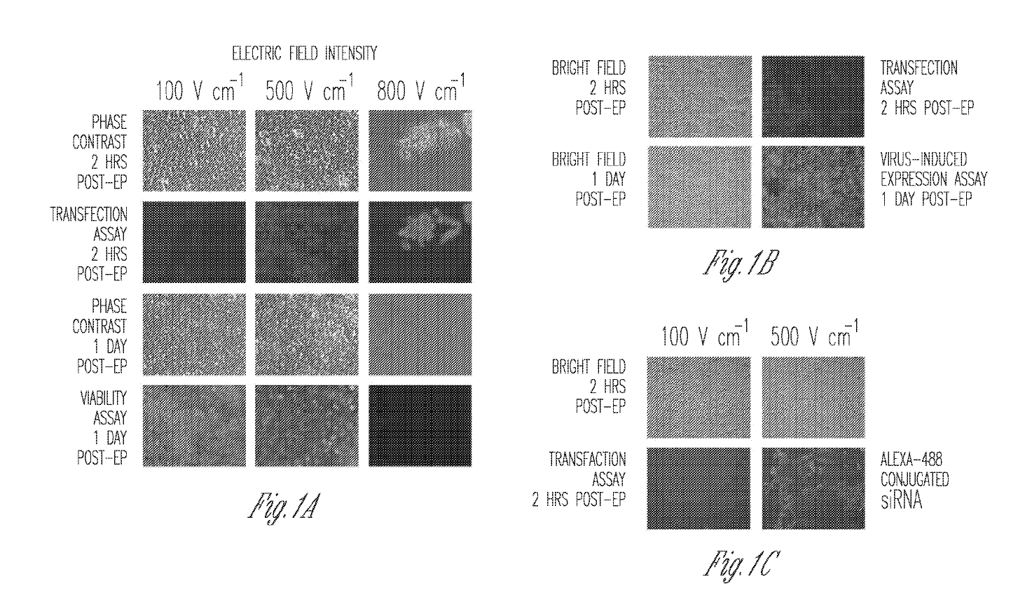

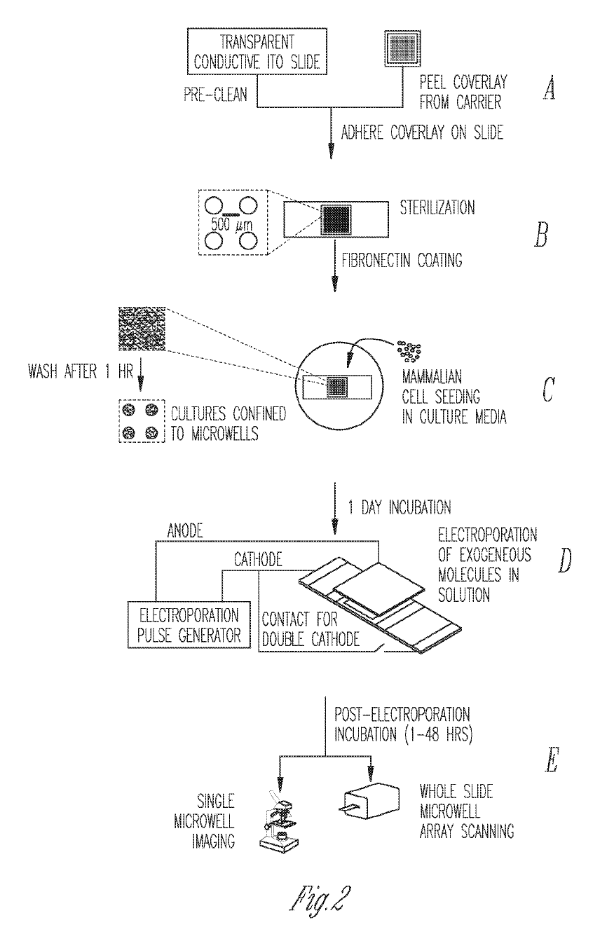

As a first step, electroporation parameters were identified that could be used to introduce exogenous molecules from solution into cells growing on transparent ITO with high efficiency and minimal loss of cell viability. A variety of electroporation parameters (differential voltage, pulse-width and number of pulses) were optimized for electroporation of propidium iodide into HEK 293T cells cultured on ITO coated glass substrates diced into pieces (protocol and electroporation setup shown in FIGS. 8A,B). Propidium Iodide is a membrane impermeant DNA stain that is excluded from cells refractory to entry of exogenous molecules. Staining with propidium iodide is an indication that the cell has become receptive to entry of molecules from outside the cell that would generally be excluded. To confirm that propidium iodide staining is not simply a consequence of cell permeability due to cell death, electroporated cells also underwent a viral infection viability assay. Electroporated cells were incubated with a lentivirus encoding green fluorescent protein GFP (viral-GFP particles) for 24 hr. Live cells (not lysed by electroporation) are expected to maintain their integrity, be permissive to viral infection, and express GFP. Because viral infection, integration, and transgene expression (in this case GFP) are dependent on the cellular machinery of its host, this is a sensitive assay to evaluate the health of electroporated cells. FIG. 1A shows three representative parameter sets with their respective transfection and viability assays. The parameter set 500 V cm⁻¹, 1 ms (pulse-width) and 1 pulse resulted in optimal (>99%) transfection efficiency and high viability for electroporating propidium iodide. Incubation of transfected cells with viral-GFP particles showed that cells remained viable after electroporation (FIG. 1B). Similar experiments were conducted with an Alexa Fluor 488-conjugated siRNA and the optimal electroporation parameters resulted in >99% transfection efficiency (FIG. 1C). The protocol on ITO-glass cut pieces provides a convenient method to optimize electroporation parameter sets that can be repeated if the cell type, exogenous molecule or other factors are changed, as is shown for human epithelial carcinoma (HeLa) cells (FIG. 9). Certain electroporation parameter sets caused the ITO substrates to brown at the surface and were avoided (FIGS. 8C,D).

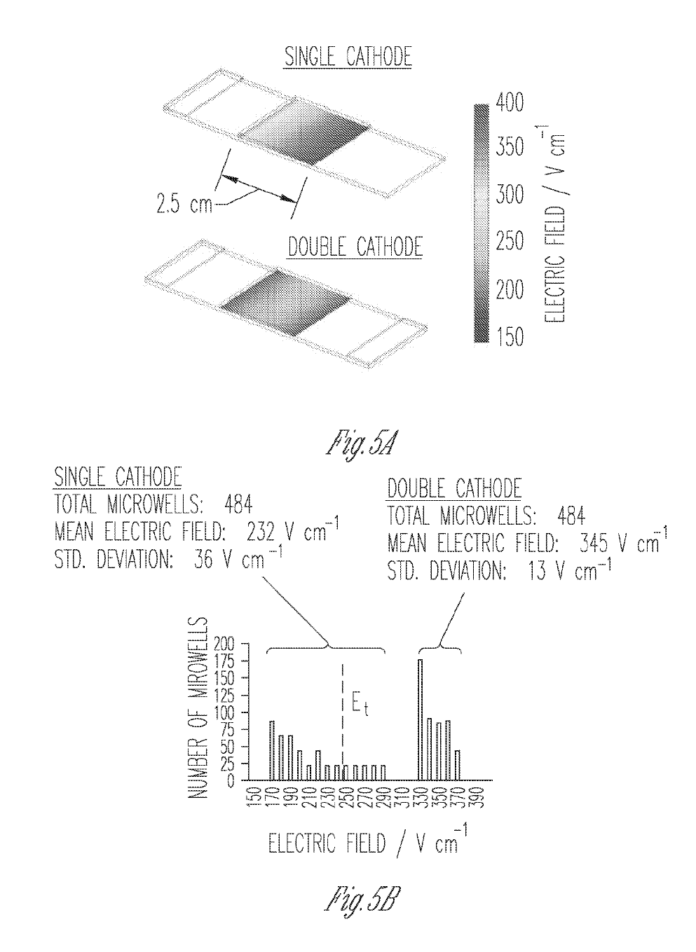

Different cell types required different electroporation parameters. The main parameters used for optimizing electroporation were electric field, pulse width and number of pulses. Initial studies with HEK293T cells (FIGS. 1, 3 and 5) were obtained by testing out several combinations of the three above-identified parameters. The general strategy was to modify the electric field and/or pulse-width, keeping the number of pulses fixed, to minimize the parametric space. The number of pulses was kept constant at one pulse and the electric field and pulse-width optimized for HEK 293T cells. The same parameters did not give high electroporation efficiency for HeLa cells. Further experimentation with electric field/pulse width combinations (keeping pulse number fixed at one pulse) gave higher electroporation efficiency while maintaining high viability (FIG. 9). However, when electroporation in primary mouse macrophages was attempted, changing the electric field of pulse width did not efficiently increase the transfection efficiency from the original parameters found for HEK 293T or HeLa cells. For the macrophages the number of pulses was then varied along with the two parameters to induce repetitive electroporation (FIG. 4) and achieve high electroporation efficiency.

Cellular Cultures within a Microwell Array

Microwells offer numerous advantages for a miniaturized genomic screening platform: they can provide a physical marker for imaging and a barrier for microscale cultures to be contained. To evaluate the prospects of utilizing microwells in a high-throughput screening platform, a simple method was used to create microwell arrays on ITO coated glass slides using laser-cut coverlays (FIGS. 2A,B). Microwells were 500 μm in diameter and separated at 1 mm inter-well distance. These dimensions were chosen to ensure that enough cells can be accommodated per microwell to assess phenotypes with statistical power. Microscale cultures were then obtained within a 484-microwell array by flooding the array in a tissue culture dish and washing away unbound cells (FIG. 2C). The cells were subsequently electroporated and analyzed (FIGS. 2 D,E). Prior to electroporation, phase contrast and cell viability (assessed with the vital dye Calcein AM) images of HEK 293T cells cultured within the microwell array were taken 24 hr post-seeding (FIG. 3A). All 484 microwells of the array had a similar degree of confluence, indicating that this approach to seed and culture cells in microwells is a robust method for obtaining uniform cell density. To assess the compatibility of the microwells with sensitive cell types such as primary cells, primary mouse macrophages were obtained and seeded within the microwell array. These cells adapted to the microwells with ease; no macrophage activation was seen (FIG. 4A).

The first step of phenotypic evaluation after a screen is completed is usually the identification of the spatial location of the cellular culture of interest on the substrate. For a miniaturized platform consisting of arrayed microscale cultures, the use of microwells may enhance the accuracy of image analysis and phenotype deconvolution. Imaging system stages are usually pre-programmed¹⁰ to the exact location of the microscale cultures on the substrate, which leaves the task of identifying the spatial location of cellular cultures to the image analysis step. Considering equipment errors and tolerances, in the absence of physical markers, image analysis can at best estimate the location of the microscale cultures. With a physical marker, in this case the edge of the microwells, the spatial location of the microscale cultures can be identified with certainty (FIG. 3C). The software first identifies the edges of the microwells, which in turn determines the center of the microscale culture position. The software then crops out the microscale culture removing the unwanted microwell edges. With subsequent modules for thresholding, segmentation, and object identification, a variety of phenotypic analysis can be accomplished (in this example simply identifying 890 electroporated cells within a microwell). The presence of the microwells dramatically enhances the ability to precisely identify the microscale cellular cultures on the substrate, thus increasing the accuracy of phenotypic annotation.

Electroporation within Microwells

Having obtained electroporation parameters for high transfection efficiency of exogenous molecules from solution into adherent cells on ITO slides, and having tested the feasibility of growing cellular microscale cultures within microwells created on this substrate, in-situ electroporation within microwells was then evaluated. HEK 293T cells growing within microwells were electroporated with three different exogenous molecules: propidium iodide, siRNA and plasmid DNA encoding GFP (FIG. 3B). Electroporation of HEK 293T cells within microwell arrays was conducted using the electroporation parameter set described above for cultures on ITO coated glass pieces without microwells (500 V cm⁻¹, 1 ms, 1 pulse) and with the exogenous molecule in solution. This resulted in transfection efficiencies >99% for propidium iodide and siRNA molecules within the microwells. It is interesting to note that the optimal electroporation parameters are essentially unchanged, even with the presence of a microwell array that effectively insulates a major region of the substrate leaving only small openings for the electroporation current to flow. This may be because the current density at the ITO-cell interface under constant voltage conditions remains essentially the same, even though the total current between cathode and anode varies due to a change of effective electrode area. With similar current density values at the interface, adherent cells in microwells should exhibit similar electroporation efficiency¹⁷, as is the case in these experiments. Mouse primary macrophages were also successfully electroporated within the microwells (FIG. 4A). A different electroporation parameter set (600 V cm⁻¹, 1 ms, 5 pulses) proved optimal for these cells. A viability assay indicated that most primary cells remained healthy post-electroporation. Microwell edge identification, microscale culture recognition, and cellular/nuclear object identification was carried out for images obtained in three distinct fluorescence channels (Hoechst: total cell nuclei count; Propidium Iodide: transfected cells; Calcein AM: viable cells). High transfection efficiency without loss of viability can be achieved in these primary cells (FIG. 4B). Measurements indicate cell viability to be 86% (for a control pulse) and 93% (for an electroporation pulse). These results indicate that electroporation itself does not induce cell death.

Highly Parallel Electroporation in a 484-microwell Array

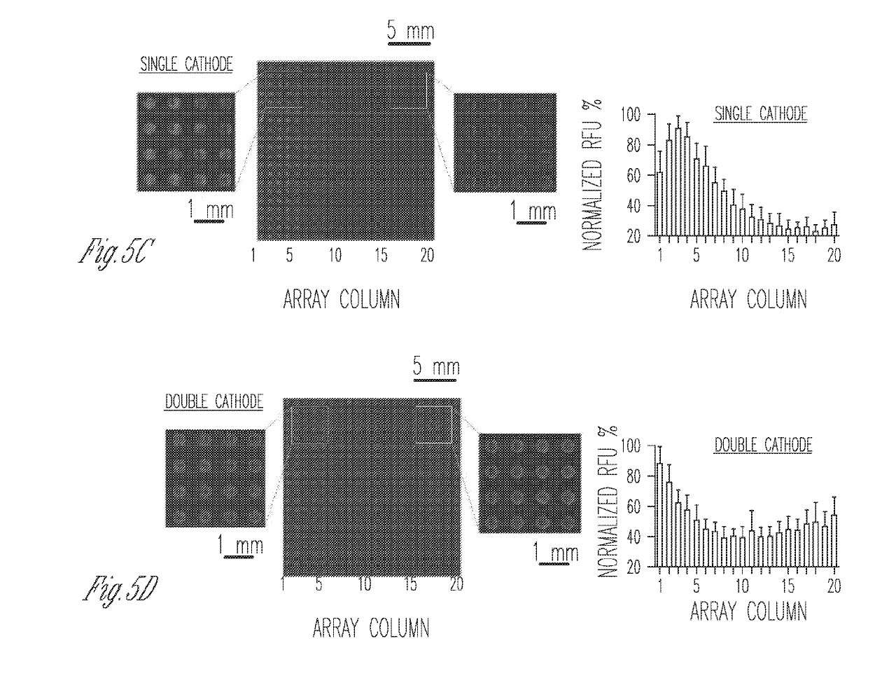

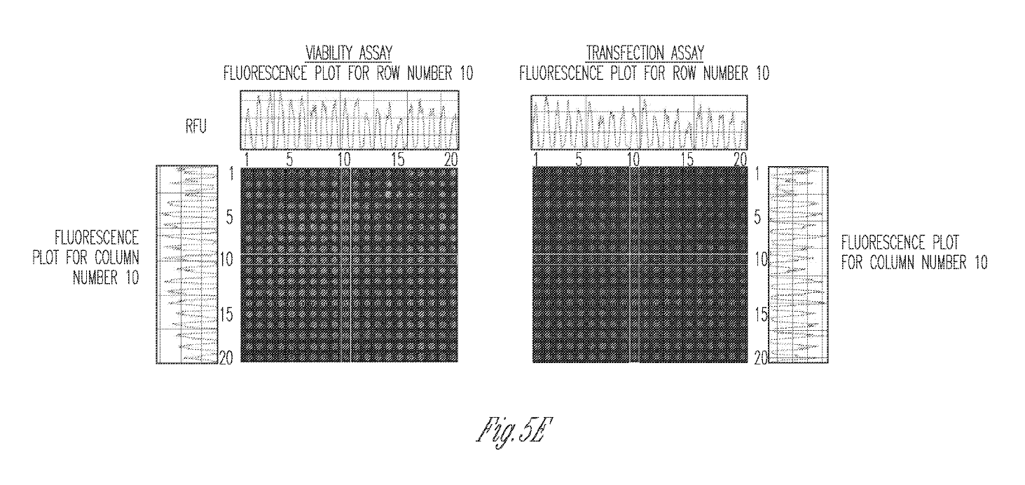

Next, the electroporation efficiency in the 484-microwell array was evaluated to determine transfection variability across the ITO coated glass slides under different cathode schemes. These slides have a surface resistivity of ˜4-8 Ω sq⁻¹, which could be high enough to ensure the surface conductivity required for electroporation, but low enough to cause noticeable voltage losses across the substrate from the point of electrode contact. Thus, it was expected that by increasing the cathode contacts on ITO, the voltage loss across the slide might be reduced. To examine this possibility, finite element analysis simulations were used to model the electric field pattern at the surface of the microwell array for a single cathode or a double cathode scheme. They predict very contrasting electric field patterns (FIG. 5A, top and bottom). With a single cathode, there is wide variation of electric field across the array (FIG. 5A, top and FIG. 5B, left graph). A double cathode scheme significantly reduces electric field variation (FIG. 5A, bottom and FIG. 5B, right graph).

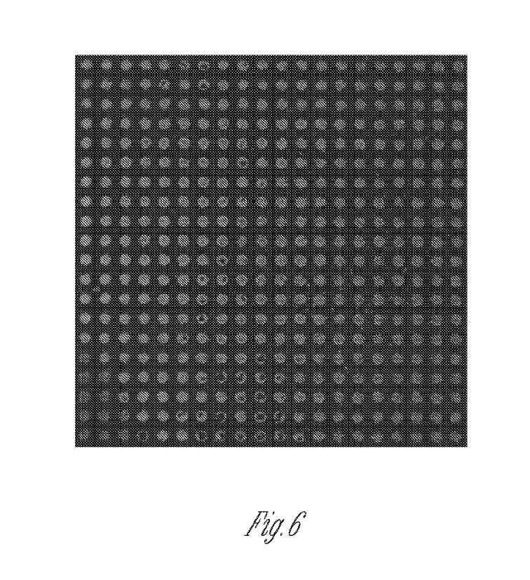

These predictions were tested in experiments conducted on a 400-microwell array (the outer rows and columns were omitted from the 484-microwell array to avoid possible edge effects) using propidium iodide as the exogenous electroporated molecule. Parallel electroporation of the microwell array with a single cathode scheme resulted in clear variation in transfection efficiency from left to right (FIG. 5C). In contrast, a double cathode scheme yielded more uniform electroporation across the microwell array (FIG. 5D). These results were applied to calculate the threshold electric field, Eₜ, necessary to obtain greater than 50% transfection of the maximum observed in any well in the microwell array. From the experimental data the column position where the efficiency dropped to ˜50% was determined (FIG. 5C, right bar graph). The Eₜ electric field value from the simulation was deemed to be ˜250 V/cm (FIG. 5A). All microwells in the double cathode scheme are expected to be above this threshold, in sharp contrast to the single cathode scheme (FIG. 5B). This prediction was empirically borne out, as most microwells in the double cathode scheme exhibited 50% or greater electroporation efficiency when compared to the maximum (FIG. 5D, right bar graph). To evaluate the ability of the modified double cathode scheme to assure uniform transfection efficiency and cellular viability across the entire 400-microwell array, transfection efficiency and cell viability were measured post-electroporation. Fluorescent intensity plots were generated to profile the changes observed for the central rows and columns for each of these assays (FIG. 5E). These results indicate that a double cathode method minimizes variation in transfection efficiency while maintaining cell viability across the entire array. Electroporation of a different molecule, Alexa Fluor 488-conjugated siRNA, was also tested within the microwell array using the double cathode method. Similar results were obtained; transfection efficiency was relatively uniform across the array (FIG. 6).

Microarraying within Microwells

Since the ultimate goal is to conduct multiplexed parallel electroporation of thousands of different molecules, a necessary component of the envisioned miniaturized high-throughput genetic screening platform will be the ability to quickly load large libraries of molecules onto the microstructure-bearing substrates. Standard microarray spotters allow microarraying of libraries (nucleic acids, proteins, and carbohydrates) from well-plates onto microscope slides with micron step resolution. These microarrayers are an excellent tool to achieve ‘world-to-chip’ ability. However, microarrayers are not usually constrained to spot within microwell structures; therefore they do not require precise alignment with pre-existing micro-sized features on the substrate. With the incorporation of microwell arrays onto the substrate, the requirement to align the microarrayer and spot precisely within individual microwells becomes a critical issue. One way to overcome this problem is to use a CCD camera in conjunction with the microarraying head to image the substrate and locate the features prior to printing. However, most standard microarrayers do not include these image-capable heads. As an alternative, a simple iterative method was developed to align microwells with the microarrayer to insure accurate spotting on the experimental substrate. First, a blank microscope slide is spotted with printing buffer using the same spotting parameters (inter-spot distance and array size) to be used on the final microwell array slide. This blank spotted slide and the microwell array-containing slide are then imaged independently in a slide scanner. Next, the two images are overlaid using image analysis software to determine X-Y alignment errors (FIG. 7A). Re-calibration of the microarrayer with these errors enables spotting to take place precisely within the microwell array. Using this approach, it was possible to consistently microarray within the center of microwells (FIG. 7B). The average size of the spots obtained was 363±17 μm, which correlates well with the manufacturer's spot size specification (365 μm) for the array pin used for spotting.

### Discussion

To fully realize the potential of genome-wide cell-based genetic screening to annotate the mammalian genome, it is imperative that a next generation screening platform be developed, one that miniaturizes the screening process, thus reducing capital and reagent costs. Ideally, such a platform should possess at least five features: 1) the genetic molecules of a library must be loaded with ease into spatially separated microscale regions on a single substrate; 2) the cells of interest must thrive in the “loaded” substrate, but their motility should be restricted to individual microscale regions; 3) cells must be transfected in an efficient, highly-parallel, uniform manner at a controllable time point; 4) the method used to introduce nucleic acids into microscale cultures should be effective for both cell lines and primary cells; 5) the substrate containing the transfected microscale cultures must be compatible with existing automated imaging systems and analysis tools to allow for seamless identification of phenotypes.

Working towards these goals, here the use of microwell arrays for parallel electroporation of exogenous molecules into microscale cultures on a single substrate was demonstrated. In these experiments, a 484-microwell array was created on a conductive and transparent ITO microscope dimension slide by bonding a laser-cut adhesive coverlay. The microwells allowed for consistent culture of mammalian cells (both primary cells and immortalized cell lines) within the array. These coverlays served as a quick and easy way to create microwell arrays for initial lab-on-a-chip experiments. Additionally, to obtain precise straight edges and defined geometric features, microfabrication techniques using photopatternable polymers were incorporated.

ITO conductive slides have previously been used to achieve efficient electroporation of exogenous molecules into mammalian cells¹⁸, but their utility in arrays of microscale cultures of the kind required to perform genome-wide genetic screens has not been explored. When the desired application is that of high-throughput screening of genetic libraries in mammalian cells, a critical requirement is to segregate both the individual nucleic acids and the cellular cultures into microscale domains on a single substrate prior to electroporation. The results herein demonstrate that it is possible to create an array of microscale cellular cultures on conductive substrates using a microwell-based approach that allows for parallel electroporation of exogenous molecules (propidium iodide, siRNAs and plasmid DNA) from solution into cells contained within the microscale domains. With a single cathode electroporation scheme, voltage drops caused by the surface resistivity of ITO resulted in a non-uniform electric field across the microwell array and variable electroporation efficiency across the substrate. One way to resolve this issue would be to set the anode at a slight angle (˜1 degree) above the cathode, but this requires precisely machined parts and spacers¹⁸. A simpler alternative is that of using simultaneous multiple contacts on the ITO cathode to reduce voltage drops across the substrate. This scheme results in uniform electroporation efficiency across the microwell array, allowing for highly parallel electroporation on a single substrate. Future designs may incorporate additional electrodes and possibly patterns of cathode on the ITO to further optimize uniformity of electric field distribution.

The use of microwells significantly enhances the ability to identify the spatial location of microscale cellular cultures and improves the image acquisition and analysis steps. Microscale cultures can also be created using purely surface chemistry techniques to create patterned regions of cell adhesion/non-adhesion¹⁴, but the lack of a physical marker makes it difficult to precisely identify the location of the cultures during imaging and analysis. Cartesian or angular shifts during imaging can complicate identification of the microscale domains and hamper downstream image analysis, obscuring phenotype evaluation. The microwell edges provide a clear physical indication of the spatial location of the cultures; they enable centering of individual microscale images during image processing and analysis. Moreover, the microwells provide physical containment for cells transfected with an individual nucleic acid, restricting migration and contamination of neighboring cellular cultures transfected with other nucleic acids. This feature may be particularly relevant for time-lapse studies, in which cells are monitored multiple times after transfection. Another advantage of microwells is that microscale cultures experience significantly lower flow shear stresses as indicated by simulations¹⁹. This may minimize cell stripping during experimental protocols that could lead to inter-spot contamination. In the future it may be possible to use a combination of microwells and surface chemistry on the plateau areas to further prevent inter-well cell motility²⁰.

The ability to transfer large libraries (e.g. genome-size) exogenous molecules swiftly and precisely is an important requirement for a miniaturized high-throughput screening platform. Contact pins or non-contact pin-less spotters offer a potential approach to transfer libraries of genetic molecules from stock well plates to the miniaturized platform. However, instrument and substrate edge tolerances make it challenging to ensure high-precision spotting of molecules within microscale regions and geometric microstructures such as the microwells described herein. To address this issue, a simple iterative process of imaging the microwell array and re-calibrating the microarrayer using overlaid images of blank slide prints that enables accurate spotting of genetic molecules within the microwell arrays was developed. Here, the ability to achieve parallel electroporation of exogenous molecules in solution into cells contained in a 484-microwell array was developed. Maintaining similar microwell dimensions, and introducing changes to the microwell array design to scale the technology, it will be possible to conduct a genome-wide screen (˜25,000 molecules)²¹ in mammalian cells in a single substrate the size of a 96-well plate.

In summary, conditions have been found to achieve parallel introduction of exogenous molecules into primary and immortalized mammalian cells cultured within a 484-microwell array created on an ITO slide. The microwell array allows for the consistent generation of segregated microscale cultures. The microwell edges enable precise identification of the spatial location of the microscale cultures during image analysis. Finally, these microwell arrays are fully compatible with standard microarraying equipment, allowing swift transfer of nucleic acid libraries from stock plates onto the miniaturized platform. These advances are the basis for the miniaturized high-throughput genetic screening platform for mammalian cells described herein.

### Example Embodiments

Example 1 includes an apparatus for use in introducing an exogenous molecule into a cell. The apparatus includes a substrate, an electrode layer, and a walled portion. The electrode layer is disposed on a first side of the substrate and is composed of an electrically conductive material. The walled portion is disposed on the first side of the substrate. The walled portion includes a plurality of walls forming a plurality of apertures, wherein the walled portion and the substrate form a plurality of wells with the walls as a side of the wells and the substrate as a bottom of the wells. The walls of the walled portion substantially align with the electrode layer.

In Example 2, the subject matter of Example 1 can optionally include wherein the walled portion overlays the electrode layer.

In Example 3, the subject matter of any one of Examples 1-2 can optionally include wherein the substrate comprises a base portion, and an electrically conductive material portion bonded to the base portion, wherein the electrode layer and the walled portion are disposed on the electrically conductive material portion of the substrate.

In Example 4, the subject matter of any one of Examples 1-3 can optionally include wherein the electrode layer is contained within the walled portion such that the electrically conductive material of the electrode layer is not exposed within a well.

In Example 5, the subject matter of any one of Examples 1-4 can optionally include wherein the electrode layer has a greater electrical conductivity than the electrically conductive material portion of the base portion.

In Example 6, the subject matter of any one of Examples 1-5 can optionally include wherein the electrode layer forms a grid having a plurality of apertures that substantially surround the wells, wherein the plurality of apertures align with the plurality of apertures in the walled portion.

In Example 7, the subject matter of any one of Examples 1-2 can include wherein the substrate is composed of an electrically non-conductive material and wherein the electrode layer includes a first portion and a second portion that is not electrically coupled to the first portion, wherein the electrode layer is partially covered by the walled portion such that a portion of the first portion of the electrode layer is exposed within a well and a portion of the second portion of the electrode layer is exposed within the well.

In Example 8, the subject matter of any one of Examples 1-2 and 7 can optionally include wherein the electrode layer comprises a plurality of parallel lines and the walls of the walled portion are aligned with the parallel lines.

In Example 9, the subject matter of any one of Examples 1-2 and 7-8 can optionally include wherein alternating lines of the parallel lines are not electrically coupled to one another.

Example 10 includes a method of fabricating a microwell array. The method includes placing a electrically conductive layer on a substrate and patterning the electrically conductive layer to form an electrode layer. The method also includes placing a photo-resist material over the conductive layer on the substrate, and patterning the photoresist material to form a walled portion on the substrate. The walled portion includes a plurality of walls forming a plurality of apertures, wherein the walled portion and the substrate form a plurality of wells with the walls as a side of the wells and the substrate as a bottom of the wells, and wherein the walls of the walled portion substantially align with the electrode layer.

In Example 11, the subject matter of Example 10 can optionally include wherein the conductive layer is patterned using photo-lithography and wherein the photo-resist material is patterned using photo-lithography.

In Example 12, the subject matter of any one of Examples 10-11 can optionally include wherein the substrate include a base material portion and an electrically conductive material portion, wherein the electrically conductive layer is placed on the conductive material portion.

In Example 13, the subject matter of any one of Examples 10-12 can optionally include wherein the electrode layer is contained within the walled portion such that the electrode layer is not exposed within a well.

In Example 14, the subject matter of any one of Examples 10-11 can optionally include wherein the substrate is composed of an electrically non-conductive material and wherein the electrode layer includes a first portion and a second portion that is not electrically coupled to the first portion, wherein the electrode layer is partially covered by the walled portion such that a portion of the first portion of the electrode layer is exposed within a well and a portion of the second portion of the electrode layer is exposed within the well.

Example 15 includes a method to introduce an exogenous molecule into a cell. The method includes adding the exogenous molecule and the cell to a well of the apparatus of Example 1, and introducing the exogenous molecule into the cell by electroporation.

In Example 16, the subject matter of Example 15 can optionally include wherein the exogenous molecule is mixed with a controlled release agent before addition to the well to facilitate the controlled release of the molecule in the well prior to electroporation.

In Example 17, the subject matter of any one of Examples 15-16 can optionally include wherein the exogenous molecule is added to the well before the cell is added to the well.

In Example 18, the subject matter of any one of Examples 15-17 can optionally include wherein the cell is added to the well before the exogenous molecule is added to the well.

In Example 19, the subject matter of any one of Examples 15-18 can optionally include wherein the exogenous molecule is screened for its ability to modify a characteristic of the cell after electroporation into the cell.

In Example 20, the subject matter of any one of Examples 15-19 can optionally include wherein the exogenous molecule is screened by steps comprising determining the effects of the exogenous molecule on the cell; comparing the effects to the effects of a second exogenous molecule introduced into a second cell; and selecting the exogenous molecule based on its effects on the cell.

In Example 21, the subject matter of any one of Examples 15-20 can optionally include wherein the modification of the cell is an increase in the characteristic.

In Example 22, the subject matter of any one of Examples 15-21 can optionally include wherein the modification of the cell is a decrease in the characteristic.

In Example 23, the subject matter of any one of Examples 15-22 can optionally include wherein the characteristic of the cell is its phenotype.

In Example 24, the subject matter of any one of Examples 15-23 can optionally include wherein the characteristic of the cell is apoptosis.

In Example 25, the subject matter of any one of Examples 15-24 can optionally include wherein the characteristic of the cell is expression of a gene.

In Example 26, the subject matter of any one of Examples 15-25 can optionally include wherein the exogenous molecule is selected from the group consisting of an amino acid, a polypeptide, a nucleic acid, RNA, DNA, a virus, a drug, and a nanoparticle.

In Example 27, the subject matter of any one of Examples 15-26 can optionally include wherein the cell is a prokaryotic cell or a eukaryotic cell.

In Example 28, the subject matter of any one of Examples 15-27 can optionally include wherein the cell is selected from the group consisting of a bacterial cell, an insect cell, a fungal cell, a plant cell, and a mammalian cell.

### BIBLIOGRAPHY

1. N. Perrimon and B. Mathey-Prevot, Genetics, 2007, 175, 7-16.
2. J. Moffat and D. M. Sabatini, Nat Rev Mol Cell Biol, 2006, 7, 177-187.
3. G. J. Hannon and J. J. Rossi, Nature, 2004, 431, 371-378.
4. S. M. Elbashir, J. Harborth, W. Lendeckel, A. Yalcin, K. Weber and T. Tuschl, Nature, 2001, 411, 494-498.
5. A. Fire, S. Xu, M. K. Montgomery, S. A. Kostas, S. E. Driver and C. C. Mello, Nature, 1998, 391, 806-811.
6. C. Y. Cho, S. H. Koo, Y. Wang, S. Callaway, S. Hedrick, P. A. Mak, A. P. Orth, E. C. Peters, E. Saez, M. Montminy, P. G. Schultz and S. K. Chanda, Cell Metab, 2006, 3, 367-378.
7. D. R. Rines, M. A. Gomez-Ferreria, Y. Zhou, P. DeJesus, S. Grob, S. Batalov, M. Labow, D. Huesken, C. Mickanin, J. Hall, M. Reinhardt, F. Natt, J. Lange, D. J. Sharp, S. K. Chanda and J. S. Caldwell, Genome Biology, 2008, 9,-.
8. J. Ziauddin and D. M. Sabatini, Nature, 2001, 411, 107-110.
9. B. Lehner and A. G. Fraser, Nat Methods, 2004, 1, 103-104.
10. S. Mousses, N. J. Caplen, R. Cornelison, D. Weaver, M. Basik, S. Hautaniemi, A. G. Elkahloun, R. A. Lotufo, A. Choudary, E. R. Dougherty, E. Suh and O. Kallioniemi, Genome research, 2003, 13, 2341-2347.
11. D. B. Wheeler, A. E. Carpenter and D. M. Sabatini, Nature genetics, 2005, 37 Suppl, S25-30.
12. J. M. Silva, H. Mizuno, A. Brady, R. Lucito and G. J. Hannon, Proc Natl Acad Sci USA, 2004, 101, 6548-6552.
13. K. Kato, K. Umezawa, M. Miyake, J. Miyake and T. Nagamune, Biotechniques, 2004, 37, 444-448, 450, 452.
14. F. Yamauchi, K. Kato and H. Iwata, Langmuir, 2005, 21, 8360-8367.
15. W. G. Lee, U. Demirci and A. Khademhosseini, Integrative Biology, 2009, 1, 242-251.
16. V. Molteni, X. Li, J. Nabakka, F. Liang, J. Wityak, A. Koder, L. Vargas, R. Romeo, N. Mitro, P. A. Mak, H. M. Seidel, J. A. Haslam, D. Chow, T. Tuntland, T. A. Spalding, A. Brock, M. Bradley, A. Castrillo, P. Tontonoz and E. Saez, J Med Chem, 2007, 50, 4255-4259.
17. T. Jain and J. Muthuswamy, Lab Chip, 2007, 7, 1004-1011.
18. L. Raptis and K. L. Firth, DNA Cell Biol, 1990, 9, 615-621.
19. H. C. Moeller, M. K. Mian, S. Shrivastava, B. G. Chung and A. Khademhosseini, Biomaterials, 2008, 29, 752-763.
20. M. Ochsner, M. R. Dusseiller, H. M. Grandin, S. Luna-Morris, M. Textor, V. Vogel and M. L. Smith, Lab on a Chip, 2007, 7, 1074-1077.
21. C. I. Human Genome Sequencing, Nature, 2004, 431, 931-945.
22. E. J. Moore, M. Curtin, J. Ionita, A. R. Maguire, G. Ceccone and P. Galvin, Anal Chem, 2007, 79, 2050-2057.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. However, examples in which only those elements shown and described are contemplated.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

Method examples described herein can be machine or computer-implemented, at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

Reference will now be made in detail to certain embodiments of the disclosed subject matter, examples of which are illustrated in the accompanying descriptions. While the disclosed subject matter will be described in conjunction with the enumerated claims, it will be understood that they are not intended to limit the disclosed subject matter to those claims. On the contrary, the disclosed subject matter is intended to cover all alternatives, modifications and equivalents, which may be included within the scope of the presently disclosed subject matter as defined by the claims.

What is claimed is:

1. An apparatus for use in introducing an exogenous molecule into a cell, the apparatus comprising:
a substrate;
an electrode layer on a first side of the substrate, the electrode layer composed of an electrically conductive material; and
a walled portion on the first side of the substrate, the walled portion including a plurality of walls forming a plurality of apertures, wherein the walled portion and the substrate form a plurality of wells with the walls as a side of the wells and the substrate as a bottom of the wells, and wherein the walls of the walled portion substantially align with the electrode layer.

2. The apparatus of claim 1, wherein the walled portion overlays the electrode layer.

3. The apparatus of claim 1, wherein the substrate comprises:
a base portion; and
an electrically conductive material portion bonded to the base portion, wherein the electrode layer and the walled portion are disposed on the electrically conductive material portion of the substrate.

4. The apparatus of claim 3, wherein the electrode layer is contained within the walled portion such that the electrically conductive material of the electrode layer is not exposed within a well.

5. The apparatus of claim 3, wherein the electrode layer has a greater electrical conductivity than the electrically conductive material portion of the base portion.

6. The apparatus of claim 3, wherein the electrode layer forms a grid having a plurality of apertures that substantially surround the wells, wherein the plurality of apertures align with the plurality of apertures in the walled portion.

7. The apparatus of claim 1, wherein the substrate is composed of an electrically non-conductive material and wherein the electrode layer includes a first portion and a second portion that is not electrically coupled to the first portion, wherein the electrode layer is partially covered by the walled portion such that a portion of the first portion of the electrode layer is exposed within a well and a portion of the second portion of the electrode layer is exposed within the well.

8. The apparatus of claim 7, wherein the electrode layer comprises a plurality of parallel lines and the walls of the walled portion are aligned with the parallel lines.

9. The apparatus of claim 8, wherein alternating lines of the parallel lines are not electrically coupled to one another.

10. A method of fabricating a microwell array, the method comprising:
placing a electrically conductive layer on a substrate;
patterning the electrically conductive layer to form an electrode layer;
placing a photo-resist material over the conductive layer on the substrate;
patterning the photoresist material to form a walled portion on the substrate, the walled portion including a plurality of walls forming a plurality of apertures, wherein the walled portion and the substrate form a plurality of wells with the walls as a side of the wells and the substrate as a bottom of the wells, and wherein the walls of the walled portion substantially align with the electrode layer.

11. The method of claim 10, wherein the conductive layer is patterned using photo-lithography and wherein the photo-resist material is patterned using photo-lithography.

12. The method of claim 10, wherein the substrate include a base material portion and an electrically conductive material portion, wherein the electrically conductive layer is placed on the conductive material portion.

13. The method of claim 12, wherein the electrode layer is contained within the walled portion such that the electrode layer is not exposed within a well.

14. The method of claim 10, wherein the substrate is composed of an electrically non-conductive material and wherein the electrode layer includes a first portion and a second portion that is not electrically coupled to the first portion, wherein the electrode layer is partially covered by the walled portion such that a portion of the first portion of the electrode layer is exposed within a well and a portion of the second portion of the electrode layer is exposed within the well.

15. A method to introduce an exogenous molecule into a cell, comprising:
adding the exogenous molecule and the cell to a well of the apparatus of claim 1, and
introducing the exogenous molecule into the cell by electroporation.

16. The method of claim 15, wherein the exogenous molecule is mixed with a controlled release agent before addition to the well to facilitate the controlled release of the molecule in the well prior to electroporation.

17. The method of claim 15, wherein the exogenous molecule is added to the well before the cell is added to the well.

18. The method of claim 15, wherein the cell is added to the well before the exogenous molecule is added to the well.

19. The method of claim 15, wherein the exogenous molecule is screened for its ability to modify a characteristic of the cell after electroporation into the cell.

20. The method of claim 19, wherein the exogenous molecule is screened by steps comprising:
determining the effects of the exogenous molecule on the cell;
comparing the effects to the effects of a second exogenous molecule introduced into a second cell; and
selecting the exogenous molecule based on its effects on the cell.

21. The method of claim 19, wherein the modification of the cell is an increase in the characteristic.

22. The method of claim 19, wherein the modification of the cell is a decrease in the characteristic.

23. The method of claim 19, wherein the characteristic of the cell is its phenotype.

24. The method of claim 19, wherein the characteristic of the cell is apoptosis.

25. The method of claim 19, wherein the characteristic of the cell is expression of a gene.

26. The method of claim 15, wherein the exogenous molecule is selected from the group consisting of an amino acid, a polypeptide, a nucleic acid, RNA, DNA, a virus, a drug, and a nanoparticle.

27. The method of claim 15, wherein the cell is a prokaryotic cell or a eukaryotic cell.

28. The method of claim 15, wherein the cell is selected from the group consisting of a bacterial cell, an insect cell, a fungal cell, a plant cell, and a mammalian cell.
